(19) United States
(12) Patent Application Publication
(10) Pub. No.: US 2005/0003444 A1
(43) Pub. Date: Jan. 6, 2005

(54) Novel human polynucleotides and the polypeptides encoded thereby

(75) Inventors: Michael Nehls, The Woodlands, TX (US); Brian Zambrowicz, The Woodlands, TX (US); Arthur T. Sands, The Woodlands, TX (US)

Correspondence Address: JONES DAY, 222 EAST 41ST ST, NEW YORK, NY, 10017, US

(73) Assignee: Lexicon Genetics Incorporated

(21) Appl. No.: 10/914,037

(22) Filed: Aug. 6, 2004

Related U.S. Application Data
(63) Continuation of application No. 09/428,674, filed on Oct. 27, 1999.
(60) Provisional application No. 60/106,442, filed on Oct. 30, 1998.

(51) Int. Cl. C12Q 1/68; C07H 21/04; C12N 9/00

(52) U.S. Cl. 435/6; 435/69.1; 435/183; 435/320.1; 435/325; 530/350; 536/23.2

(57) ABSTRACT

Novel human polynucleotides are disclosed that correspond to human gene trapped sequences, or GTSs. The disclosed GTSs are useful for gene discovery and as markers for, inter alia, gene expression analysis, forensic analysis, and determining the genetic basis of human disease.

This application claims priority to U.S. Provisional Application No. 60/106,442, filed Oct. 30, 1998, which is also incorporated herein by reference for any purpose.

### 1. FIELD OF THE INVENTION

The present invention is in the field of molecular genetics. The application discloses novel nucleic acid sequences that partially define the scope of human exons that can be trapped and identified by the disclosed vectors/methods, and which are useful, inter alia, for identifying the organization of the coding regions and of the human genome.

### 2. BACKGROUND OF THE INVENTION

The Human Genome Project and privately financed ventures are currently sequencing the human genome, and the substantial completion of this milestone is expected before the year 2003. The hope is that, at the conclusion of the sequencing phase, a comprehensive representation of the human genome will be available for biomedical analysis. However, the data resulting from such efforts will largely comprise human genomic sequence of which only a fraction actually encodes expressed sequence information. Although sophisticated computer-assisted exon identification programs can be applied to such genomic sequence data, the computer predictions require verification by laboratory analysis to actually identify the coding regions of the genome. Consequently, the availability of cDNA information will significantly contribute to the value of the human genomic sequence since cDNA sequence provides a direct indication of the presence of transcribed sequences as well as the location of splice junctions. Thus, the sequencing of cDNA libraries to obtain expressed sequence tags (or ESTs) that identify exons expressed within a given tissue, cell, or cell line is currently in progress. As a consequence of these efforts, a large number of EST sequences are presently compiled in public and privately held databases. However, the present EST paradigm is inherently limited by the levels and extent of mRNA production within a given cell. A related problem is the lack of cDNA sources from specific tissue and developmental expression profiles. In addition, some genes are typically only active under certain physiological conditions or are generally expressed at levels below or near the threshold necessary for cDNA cloning and detection and are therefore not effectively represented in current cDNA libraries.

Researchers have partially addressed these issues by using phage vectors to clone genomic sequences such that internal exons are trapped (Nehls, et al., 1994, Current Biology, 4(1):983-989, and Nehls, et al., 1994, Oncogene, 9:2169-2175). However, such libraries require the random cloning of genomic DNA into a suitable cloning vector in vitro, followed by reintroduction of the cloned DNA in vivo in order to express and splice the cloned genes prior to producing the cDNA library. Additionally, such methods can only “trap” the internal exons of genes. Consequently, genes containing a single exon or a single intron are typically not trapped by traditional methods of exon trapping.

### 3. SUMMARY OF THE INVENTION

The subject invention provides numerous isolated and purified novel human cDNAs produced using gene trap technology. The novel human gene trapped sequences (GTSs) of the subject invention are disclosed as SEQ ID NOS:9-1008 in the appended Sequence Listing.

The subject invention further contemplates the use of one or more of the subject GTSs, or portions thereof, to isolate cDNAs, genomic clones, or full-length genes/polynucleotides, or homologs, heterologs, paralogs, or orthologs thereof, that are capable of hybridizing to one or more of the disclosed GTSs or their complementary sequences under stringent conditions.

The subject invention additionally contemplates methods of analyzing biopolymer (e.g., oligonucleotides, polynucleotides, oligopeptides, peptides, polypeptides, proteins, etc.) sequence information comprising the steps of loading a first biopolymer sequence into or onto an electronic data storage medium (e.g., digital or analogue versions of electronic, magnetic, or optical memory, and the like) and comparing said first sequence to at least a portion of one of the polynucleotide sequences, or amino acid sequence encoded thereby, that is first disclosed in, or otherwise unique to, SEQ ID NOS:9-1008. Typically, the polynucleotide sequences, or amino acid sequences encoded thereby, will also be present on, or loaded into or onto a form of electronic data storage medium, or transferred therefrom, concurrent with or prior to comparison with the first polynucleotide.

Another embodiment of the invention is the use of a oligonucleotide or polynucleotide sequence first disclosed in at least a portion of at least one of the GTS sequences of SEQ ID NOS:9-1008 as a hybridization probe. Of particular interest is the use of such sequences in conjunction with a solid support matrix/substrate (resins, beads, membranes, plastics, polymers, metal or metallized substrates, crystalline or polycrystalline substrates, etc.). Of particular note are spatially addressable arrays (i.e., gene chips, microtiter plates, etc.) of polynucleotides wherein at least one of the polynucleotides on the spatially addressable array comprises an oligonucleotide or polynucleotide sequence first disclosed in at least one of the GTS sequences of SEQ ID NOS:9-1008.

Similarly, one or more oligonucleotide probes based on, or otherwise incorporating, sequences first disclosed in any one of SEQ ID NOS:9-1008, can be used in methods of obtaining novel gene sequence via the polymerase chain reaction or by cycle sequencing. Similar oligonucleotide hybridization probes can also comprise sequence that is complementary to a portion of a sequence that is first disclosed in, or preferably unique to, at least one of the GTS polynucleotides in the sequence listing. The oligonucleotide probes will generally comprise between about 8 nucleotides and about 80 nucleotides, preferably between about 15 and about 40 nucleotides, and more preferably between about 20 and about 35 nucleotides.

Moreover, an oligonucleotide or polynucleotide sequence first disclosed in at least one of the GTS sequences of SEQ ID NOS:9-1008 can be incorporated into a phage display system that can be used to screen for proteins, or other ligands, that are capable of binding an amino acid sequence encoded by an oligonucleotide or polynucleotide sequence first disclosed in at least one of the GTS sequences of SEQ ID NOS:9-1008.

An additional embodiment of the present invention is a library comprising individually isolated linear DNA molecules corresponding to at least a portion of the described human GTSs which are useful for synthesizing physically contiguous sequences of overlapping GTSs by, for example, the polymerase chain reaction (PCR).

The subject invention also provides for an antisense molecule which comprises at least a portion of sequence that is first disclosed in, or preferably unique to, at least one of the GTS polynucleotides.

The subject invention also contemplates a purified polypeptide in which at least a portion of the polypeptide is encoded by, and thus first disclosed by, at least a portion of a GTS of the present invention. The invention also relates to naturally occurring polynucleotides comprising the disclosed GTSs that are expressed by promoter elements other than the promoter elements that normally express the GTSs in human cells (i.e., gene activated GTSs). Such promoter elements can be directly incorporated into the cellular genome or recombinantly engineered upstream from at least a portion of a GTS (preferably at least about 50, more preferably at least about 75, and most preferably at least about 100 to 130 base in length) of the present invention, or a complement thereof. A particularly preferred embodiment includes recombinantly engineered expression vectors that similarly have or incorporate at least a, preferably unique, portion of the disclosed GTSs or complement thereof.

### 4. DESCRIPTION OF THE SEQUENCE LISTING AND FIGURES

The Sequence Listing is a compilation of nucleotide sequences obtained by sequencing a human gene trap library that at least partially identifies the genes in the target cell genome that can be trapped by the described gene trap vectors (i.e., the repertoire of genes that are active or have not been inactivated).

FIGS. 1A-1D. FIG. 1A illustrates a retroviral vector that can be used to practice the described invention. FIG. 1B shows a schematic of how a typical cellular genomic locus is effected by the integration of the retroviral construct into intronic sequences of the cellular gene. FIG. 1C shows the chimeric transcripts produced by the gene trap event as well as the locations of the binding sites for PCR primers. FIG. 1D shows how the PCR amplified cDNAs are directionally cloned into a suitable GTS vector.

### 5. DETAILED DESCRIPTION OF THE INVENTION

The present invention is directed to novel human polynucleotide sequences obtained from cDNA libraries generated by the normalized expression of genomic exons using gene trap technology. In particular, the disclosed novel polynucleotides were generated using a modified reverse-orientation retroviral gene trap vector that was nonspecifically integrated into the target cell genome, although other polynucleotide (DNA or RNA) gene trap vectors could have been introduced to the target cells by, for example, transfection, electroporation, or retrotransposition. Preferred retroviral vectors that can be used to practice the present invention (as well as methods and recombinant tools for making and using the described GTSs) are disclosed in, inter alia, U.S. application Ser. No. 09/276,533, filed Mar. 25, 1999 which is herein incorporated by reference in its entirety.

After integration, the exogenous promoter of the sequence acquisition, or 3′ gene trap, component of the vector was used to express and splice a chimeric mRNA that was subsequently reverse transcribed, amplified, and subject to DNA sequence analysis. Unlike conventional cDNA libraries, the presently disclosed libraries are largely unaffected by the bias inherent in cDNA libraries that rely solely on endogenous mRNA expression. Additionally, by integrating a vector into the target cell genes, a chimeric mRNA is produced that allows for the specific expansion and isolation of cDNAs corresponding to the chimeric mRNAs using vector specific primers.

As used herein the term “gene trapped sequence”, or “GTS”, refers to nucleotide sequences that correspond to naturally occurring endogenously encoded human exons that have been expressed as part of a chimeric “gene trapped” mRNA. Typically, the chimeric mRNA incorporates at least a portion of sequence that has been engineered into the sequence acquisition exon of a gene trap vector which, inter alia, facilitates cDNA production by reverse transcriptase and amplification of the cDNA by PCR to produce an isolated linear DNA molecule. The disclosed GTSs do not include vector encoded sequences.

The term “GTS” not only refers to polynucleotides that are exactly complementary to naturally occurring human mRNA, but also refers to “GTS derivatives”. The term “GTS derivative” also refers to heterologs, paralogs, orthologs, and allelic variants of the specific GTSs described herein. In addition, a GTS may include the complete coding region for a naturally occurring peptide or polypeptide. A GTS may also include a complete open reading frame.

The term “GTS peptide” as used herein includes oligopeptides or polypeptides sharing biological activity and/or immunogenicity (or immunological cross-reactivity) with an amino acid sequence encoded by at least one of the disclosed GTSs or complement thereof. The terms “biological activity” (or “biological characteristics”) of a polypeptide refers to the structural or biochemical function of the polypeptide in the normal biological processes of the organism in which the polypeptide naturally occurs. Examples of such characteristics include protein structure and/or conformation, which can be determined biochemically by reaction with appropriate ligands or receptors or by suitable biological assays.

A GTS peptide may also correspond to a full-length naturally occurring peptide or polypeptide. GTS peptides can have amino acid sequences that directly correspond to naturally occurring polypeptides or amino acid sequences or can comprise minor variations. Such variations can include amino acid substitutions that are the result of the replacement of one amino acid with another amino acid having a similar structural and/or chemical properties, such as the substitution of a leucine with an isoleucine or valine, an aspartate with a glutamate, or a threonine with a serine, i.e., conservative amino acid replacements. Additional variations include minor amino acid deletions and/or insertions, typically in the range of about 1 to 6 amino acids, and can also include one or more amino acid substitutions. Guidance in determining which GTS peptide amino acid residues can be replaced or deleted without abolishing the biological activity of interest may be determined empirically, or by using computer amino acid sequence databases to identify polypeptides that are homologous to a given GTS peptide and trying to avoid amino acid substitutions in conserved regions of homology.

“Homology” refers to the similarity or the degree of similarity between a reference, or known polynucleotide and/or polypeptide and a test nucleotide sequence and/or its orresponding amino acid sequence. As used herein, “homology” is defined by sequence imilarity between a reference sequence and at least a portion of the newly sequenced nucleotide. Typically, a corresponding amino acid sequence similarity should exist between the peptides encoded by such homologous sequences.

To determine whether proteins are homologous, the GTS sequence is translated into the corresponding amino acid sequence. The amino acid sequence is then compared with reference polypeptide sequences. A short string of matching amino acid sequence can constitute good evidence of homology (for example, repeating Gly-Pro-X sequence, or the presence of an RGD motif). However, typically a larger number of similar amino acids is required to label two sequences homologous. Generally, the match needs to be at least about 7 or 8 amino acids, among which perhaps one mismatch is allowed. These criteria allow good sensitivity in finding all relevant sequences while providing a threshold amount of selectivity.

After peptide homology has been found, the respective nucleotide sequences are compared. An alignment of the reference and new sequences should show at least about 60%, and preferably at least about 65%, agreement over the minimum of 21 nucleotides which correspond to the 6 matching amino acids. Generally, a low percentage of agreement is acceptable if the differences are in the “wobble” position (or third nucleotide of the triplet coding for an amino acid).

As used herein, a “mutated” polypeptide has an altered primary structure typically resulting from corresponding mutations in the nucleotide sequence encoding the protein or polypeptide. As such, the term “mutated” polypeptides can include allelic variants. Mutational changes in the primary structure of a polypeptide result from deletions, additions or substitutions. A “deletion” is defined as a change in a polypeptide sequence in which one or more internal amino acid residues are absent. An “addition” is defined as a change in a polypeptide sequence which has resulted in one or more additional internal amino acid residues as compared to the wild type. A “substitution” results from the replacement of one or more amino acid residues by other residues. A polypeptide “fragment” is a polypeptide consisting of a primary amino acid sequence which is identical to a portion of the primary sequence of the polypeptide to which the polypeptide is related.

A host cell “expresses” a gene or DNA when the gene or DNA is transcribed into RNA that may optionally be translated to produce a polypeptide.

The subject invention also includes GTSs which are incorporated into expression vectors and transformed into host cells which subsequently express the polynucleotides and/or polypeptides encoded by the GTSs.

The subject invention also includes antibodies capable of specifically binding to GTS peptides, as well as methods of detecting a GTS peptides or the corresponding protein by combining a sample for analysis with an antibody capable of specifically binding to a GTS peptide and detecting the formation of antibody complexes present in the sample.

The subject invention also includes a method of isolating a GTS peptide, or its corresponding protein comprising the step of separating the GTS peptide, or its corresponding protein, from a solution utilizing an antibody capable of specifically binding to the GTS peptide or its corresponding protein.

The subject invention also provides for markers for use in detecting diseases, biological events, cell types and tissues which comprise at least a portion of a GTS sequence.

Further, the subject invention provides polynucleotide markers useful for physical and genetic mapping of the human, and/or certain model organism, genome(s). In particular, the nucleotide sequences in the Sequence Listing provide sequence tagged sites (STS), that will be useful in completing an STS-based physical map of the human genome, a goal of the human genome project (Collins, F. and Galas, D. (1993) Science 262:43-46). Additionally, some of these sequences will identify new genes. These new genes will be useful in completing physical and genetic maps of all the genes in the human genome, another goal of the human genome project.

The exons contained in the disclosed GTSs contain open reading frames (present in one of the three reading frames in either orientation of the sequence). Typically, the gene trap strategy employed to generate the GTS sequences allows for the directional cloning and identification of the sense strand. However, it is possible that occasional sequencing errors or random reverse transcription, or PCR aberrations will mask the presence of the appropriate open reading frame. In such cases of sequencing error, it is possible to determine the corresponding GTS sequence by expressing the GTS in an appropriate expression system and determining the amino acid sequence by standard peptide mapping and sequencing techniques (Current Protocols in Molecular Biology, John Wiley & Sons, Vol. 2, Sec 16, 1989). Additionally, the actual reading frame and amino acid sequence of a given nucleotide sequence may be determined by in vitro synthesis of a portion of an oligopeptide comprising a possible amino acid scquence and preparing antibodies to the oligopeptide. If the antibodies react with cells from which the GTS of interest was derived, the reading frame is likely correct. Alternatively, codon usage analysis can be used to track and correct reading frame shifts in gene sequence data.

The correct amino acid sequence of a GTS protein is largely a function of the DNA sequence and the correct amino acid sequence can be readily determined using routine techniques. For example, by providing independent three fold sequencing coverage of the GTS library, random sequencing/RT/PCR errors can be identified and corrected by selecting the sequence represented by the majority of gene trap sequences covering a given nucleotide.

The nucleotide sequences of the Sequence Listing may contain some sequencing errors and several of the nucleotide sequences of the Sequence Listing may contain nucleotides that have not been precisely identified, typically designated by an N, rather than A, T, C, or G. Since each of the nucleotide sequences presented in the Sequence Listing is believed to uniquely identify a novel GTS, any sequencing errors or N's in the nucleotide sequences of the Sequence Listing do not present a problem in practicing the subject invention. Several methods employing standard recombinant methodology, for example, as described in Molecular Cloning: Laboratory Manual 2nd ed., Sambrook et al. (1989), Cold Spring Harbor Laboratory, Cold Spring Harbor, N.Y. (or periodic updates thereof), may be used to correct errors and complete the missing sequence information. For example, a nucleotide and/or oligonucleotide corresponding to a portion of a nucleotide sequence of GTS of interest, can be chemically or biochemically synthesized in vitro, and used as a hybridization probe to screen a cDNA library in order to identify and obtain library isolates comprising recombinant DNA sequences containing the GTS cDNA sequence of interest. The library isolate may then be independently subjected to nucleotide sequencing using one or more standard sequencing procedures so as to obtain a complete and accurate nucleotide sequence.

For the purposes of this disclosure, the term “isolated and purified polynucleotide” comprises a polynucleotide purified from a natural cell or tissue as well as polynucleotides which are complementary to the polynucleotides isolated from the natural cell or tissue. One example of an isolated or purified polynucleotide, or a substantially isolated preparation thereof, is a preparation where the polynucleotide of interest represents at least about 80 percent, preferably at least about 85 percent, and more preferably at least about 90 to 95 percent or more of the net product(s) that can be visualized on a DNA agarose gel stained with ethidium bromide.

The described GTSs were obtained from isolates of a cDNA library. Clones isolated from cDNA libraries generated by 3′ gene trapping typically contain only a portion of the mature RNA transcript that has been spliced to a vector encoded sequence acquisition exon, and therefore such clones may only encode a portion of the polypeptide of interest (however, it should be appreciated that a number of the disclosed GTSs may encode full-length ORFs). To obtain the remainder of the sequence, the GTSs can be used as hybridization probes to re-screen the same or a different cDNA library, and additional clones isolated by the re-screening can be purified and characterized using standard methods (Benton and Davis, 1977, Science, 196:180-183). Once sufficiently purified, the size of the DNA insert can be approximated by agarose gel electrophoresis and the larger clones can be analyzed to determine the exact number of bases by DNA sequencing. Frequently, the use of a library different from the one which contained the original clone is useful for this purpose, and particularly a library that has been prepared with extra care to extend cDNA synthesis to full-length, or a library that has been intentionally primed with random primers in order to “jump over” particularly difficult regions of the transcript sequence.

Missing upstream DNA sequence can also be obtained by “primer extension” of the cDNA isolate, a practice common in the art (Sambrook et al. (1989), Molecular Cloning: Laboratory Manual 2nd ed. pg 7.79-7.83, Cold Spring Harbor Laboratory, Cold Spring Harbor, N.Y.), whereby a sequence-specific oligonucleotide is used to prime reverse-transcription near the 5′-end of the cDNA clone and the resulting product is either cloned into a bacterial vector or is analyzed directly by DNA sequencing. Finally, newer methods to extend clones in either direction employ oligonucleotide-directed thermocyclic DNA amplification of the missing sequences, wherein a combination of a cDNA-specific primer and a degenerate, vector-specific, or oligo-dT-binding second oligonucleotide can be used to prime strand synthesis. In any of the above methods or other methods of detecting additional cDNA sequence, two or more resulting clones containing the partial cDNA sequence can be recombined to form a single full-length cDNA by standard cloning methods. The resulting full-length cDNA may subsequently be transferred into any of a number of appropriate expression vectors.

In many instances, the sequencing of clones resulting from independent nonspecific gene trap events will result in a natural redundancy of sequencing more than one cDNA from a particular gene. As discussed above, this feature is a built in form of error detection and correction. These independent gene trap events can also be combined using the various overlapping regions of sequence into an entire contiguous sequence (“contig”) containing the complete nucleotide sequence of the full length cDNA. Similar methodology can be used to combine one or more GTSs with one or more publicly available, or proprietary, ESTs to synthesize, electronically or chemically, a contiguous sequence.

The ABI Assembler application, part of the INHERITS DNA analysis system (Applied Biosystems, Inc., Foster City, Calif.), creates and manages sequence assembly projects by assembling data from selected sequence fragments into a larger sequence. The Assembler combines two advanced computer technologies which maximize the ability to assemble sequenced DNA fragments into Assemblages, a special grouping of data where the relationships between sequences are shown by graphic overlap, alignment and statistical views. The process is based on the Meyers-Kececioglu model of fragment assembly (INHERITS™ Assembler User's Manual, Applied Biosystems, Inc., Foster City, Calif.), and uses graph theory as the foundation of a very rigorous multiple sequence alignment program for assembling DNA sequence fragments. Additional methods of using GTSs and obtaining full length versions thereof are discussed in U.S. Pat. No. 5,817,479, herein incorporated by reference.

It will be appreciated by those skilled in the art that as a result of the degeneracy of the genetic code (see, for example, Table 4-1 at page 109 of “Molecular Cell Biology”, 1986, J. Darnell et al. eds., Scientific American Books, New York, N.Y., herein incorporated by reference) a multitude of GTS nucleotide sequences, some bearing minimal nucleotide sequence homology to the nucleotide sequence of genes naturally encoding GTS peptides, can be produced. The invention has specifically contemplated each and every possible variation of nucleotide sequence that could be made by selecting combinations based on possible codon choices. These combinations are made in accordance with the standard triplet genetic code as applied to the nucleotide sequence of naturally occurring human GTS nucleotide sequences and all such variations are to be considered as being specifically disclosed. Once the triplet codons are “translated” (which can be done electronically) into their amino acid counterparts, the amino acid sequences encoded by the GTS ORFs effectively represent a generic representation of the various nucleotide sequences that can encode the amino acid sequence (i.e., each amino acid is generic for the various nucleotide codons that correspond to that amino acid).

The presently described novel human GTSs provide unique tools for diagnostic gene expression analysis, for cross species hybridization analysis, for genetic manipulations using a variety of techniques, like, for example, antisense inhibition, gene targeting, the identification or generation of full-length cDNA, mapping exons in the human genome, identifying exon splice junctions, gene therapy, gene delivery, chromosome mapping, etc. Furthermore, the expression-based detection and isolation of the described novel polynucleotides verifies that the genes encoding these sequences have not been inactivated by, for example, the covalent modification (methylation, acetylation, glycosylation, etc.) of the target cell genome, or inhibiting the function of transcriptional control elements. The fact that the genes have not been inactivated in the target cell genome can indicate an involvement in cellular metabolism, catabolism, homeostasis, or any of a wide variety of developmental and cell differentiation processes or the regulation of physiological or endocrine functions in the body, etc. (although treating the target cell with, for example, histone deacetylators can partially compensate for such inactivation and expand the target size of a given trapping construct). These data are especially useful when correlated with cDNA data from differentiated tissues and/or cells or cell lines in order to determine whether the absence of expression is regulated at the level of transcription or gene inactivation.

5.1 Polynucleotides of the Present Invention

The nucleotide sequences of the various isolated human GTSs of the present invention appear in the Sequence Listing as SEQ ID NOS:9-1008. Additional embodiments of the present invention are GTS variants, or homologs, paralogs, orthologs, etc., which include isolated polynucleotides, or complements thereof, that hybridize to one or more of the disclosed GTSs of SEQ ID NOS:9-1008 under stringent, or preferably highly stringent, conditions. By way of example and not limitation, high stringency hybridization conditions can be defined as follows: Prehybridization of filters containing DNA to be screened is carried out for 8 h to overnight at 65° C. in a buffer containing 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe (alternatively, as in all hybridizations described herein, approximately 42, 44, 46, 48, 50, 52, 54, 56, 58, 62, 64, 66, 68, 70, or about 72 degrees or more can be used). The filters are then washed in approximately 1× wash mix (10× wash mix contains 3M NaCl, 0.6M Tris base, and 0.02M EDTA, alternatively, as with all washes described herein, 2×, 3×, 4×, 5×, 6× wash mix, or more, can be used) twice for 5 minutes each at room temperature, then in 1× wash mix containing 1% SDS at 60° C. (alternatively, as in all washes described herein, approximately 42, 44, 46, 48, 50, 52, 54, 56, 58, 62, 64, 66, 68, 70, or about 72 degrees or more can be used) for about 30 min, and finally in 0.3× wash mix (alternatively, as in all final washes described herein, approximately, 0.2×, 0.4×, 0.6×, 0.8×, 1×, or any concentration between about 2× and about 6× can be used in conjunction with a suitable wash temperature) containing 0.1% SDS at 60° C. (alternatively, approximately 42, 44, 46, 48, 50, 52, 54, 56, 58, 62, 64, 66, 68, 70, or about 72 degrees or more can be used) for about 30 min. The filters are then air dried and exposed to x-ray film for autoradiography. In an alternative protocol, washing of filters is done for 37° C. for 1 h in a solution containing 2×SSC, 0.01% PVP, 0.01% Ficoll, and 0.01% BSA. This is followed by a wash in 0.1×SSC at 50° C. for 45 min before autoradiography. Another example of hybridization under highly stringent conditions is hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Wiley & sons, Inc., New York, at p. 2.10.3).

Preferably, such GTS variants will encode at least a portion or domain of a, preferably naturally occurring, protein or polypeptide that encodes a functional equivalent to a protein or polypeptide, or portion or domain thereof, encoded by the disclosed GTSs. Additional examples of GTS variants include polynucleotides, or complements thereof, that are capable of binding to the disclosed GTSs under less stringent conditions, such as moderately stringent conditions, (e.g., washing in 0.2×SSC/0.1% SDS at 42° C. (Ausubel et al., 1989, supra). Moderately stringent conditions can be additionally defined, for example, as follows: Filters containing DNA are pretreated for 6 h at 55° C. in a solution containing 6×SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 h at 55° C. (alternatively, as in all hybridizations described herein, approximately 42, 44, 46, 48, 50, 52, 54, 56, 58, 62, 64, 66, 68, 70, or about 72 degrees or more can be used in combination with a suitable concentration of salt). The filters are then washed in approximately 1× wash mix (10× wash mix contains 3M NaCl, 0.6M Tris base, and 0.02M EDTA, alternatively, as with all washes described herein, 2×, 3×, 4×, 5×, 6× wash mix, or more, can be used) twice for 5 minutes each at room temperature, then in 1× wash mix containing 1% SDS at 60° C. (alternatively, as in all washes described herein, approximately, 42, 44, 46, 48, 50, 52, 54, 56, 58, 62, 64, 66, 68, 70, or about 72 degrees or more can be used) for about 30 min, and finally in 0.3× wash mix (alternatively, as in all final washes described herein approximately 0.2×, 0.4×, 0.6×, 0.8×, 1×, or any concentration between about 2× and about 6× can be used in conjunction with a suitable wash temperature) containing 0.1% SDS at 60° C. (alternatively, approximately 42, 44, 45, 48, 50, 52, 54, 56, 58, 62, 64, 66, 68, 70, or about 72 degrees or more can be used) for about 30 min. The filters are then air dried and exposed to x-ray film for autoradiography.

In an alternative protocol, washing of filters is done twice for 30 minutes at 60° C. in a solution containing 1×SSC and 0.1% SDS. Filters are blotted dry and exposed for autoradiography.

Other conditions of moderate stringency which may be used are well-known in the art. For example, washing of filters can be done at 37° C. for 1 h in a solution containing 2×SSC, 0.1% SDS. Another example of hybridization under moderately stringent conditions is washing in 0.2×SSC/0.1% SDS at 42° C. (Ausubel et al., 1989, supra). Such less stringent conditions may also be, for example, low stringency hybridization conditions. By way of example and not limitation, procedures using such conditions of low stringency are as follows (see also Shilo and Weinberg, 1981, Proc. Natl. Acad. Sci. USA 78:6789-6792): Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C. (alternatively, as in all hybridizations described herein, approximately 42, 44, 46, 48, 50, 52, 54, 56, 58, 62, 64, 66, 15 68, 70, or about 72 degrees or more can be used). The filters are then washed in approximately 1× wash mix (10× wash mix contains 3M NaCl, 0.6M Tris base, and 0.02M EDTA, alternatively, as with all washes described herein, 2×, 3×, 4×, 5×, 6× wash mix, or more, can be used) twice for five minutes each at room temperature, then in 1× wash mix containing 1% SDS at 60° C. (alternatively, as in all washes described herein, approximately 42, 44, 46, 48, 50, 52, 54, 56, 58, 62, 64, 66, 68, 70, or about 72 degrees or more can be used) for about 30 min, and finally in 0.3× wash mix (alternatively, as in all final washes described herein, approximately, 0.2×, 0.4×, 0.6×, 0.8×, 1×, or any concentration between about 2× and about 6× can be used in conjunction with a suitable wash temperature) containing 0.1% SDS at 60° C. (alternatively, approximately 42, 44, 46, 48, 50, 52, 54, 56, 58, 62, 64, 66, 68, 25 70, or about 72 degrees or more can be used) for about 30 min. The filters are then air dried and exposed to x-ray film for autoradiography. In yet another alternative protocol, washing of filters is done for 1.5 h at 55° C. in a solution containing 2×SSC, 25 mM Tris-HCl (pH 7.4), 5mM EDTA, and 0.1% SDS. The wash solution is replaced with fresh solution and incubated an additional 1.5 h at 60° C. Filters are then blotted dry and exposed for autoradiography. If necessary, filters are washed for a third time at 65-68° C. and reexposed to film. Other conditions of low stringency which may be used are well known in the art (e.g., as employed for cross-species hybridizations). Preferably, GTS variants identified or isolated using the above methods will also encode a functionally equivalent gene product (i.e., protein, polypeptide, or domain thereof, encoding or otherwise associated with a function or structure at least partially encoded by the complementary GTS).

Additional embodiments contemplated by the present invention include any polynucleotide sequence comprising a continuous stretch of nucleotide sequence originally disclosed in, or otherwise unique to, any of the GTSs of SEQ ID NOS:9-1008 that are at least 8, or at least 10, or at least 14, or at least 20, or at least 30, or at least about 40, and preferably at least about 60 consecutive nucleotides up to about several hundred bases of nucleotide sequence or an entire GTS sequence. Functional equivalents of the gene products of SEQ ID NOS:9-1008 include naturally occurring variants of SEQ ID NOS:9-1008 present in other species, and mutant variants, both naturally occurring and engineered, which retain at least some of the functional activities of the gene products of SEQ ID NOS:9- 1008.

The invention also includes degenerate variants of the claimed GTS sequences, and products encoded thereby. Such variants may be 80% identical to any one of SEQ ID NOS: 9-1008, more preferably 85%, more preferably 90%, more preferably 95% and most preferably 98% identical. The degree of identity (or the degree of homology) of a polynucleotide sequence to any one of SEQ ID NOS: 9-1008 may be determined using any sequence analysis program known in the art, for example, the University of Wisconsin GCG sequence analysis package, SEQUENCHER 3.0, Gene Codes Corp., Ann Arbor, Mich. The invention further includes GTS derivatives wherein any of the disclosed GTSs, or GTS variants, is linked to another polynucleotide molecule, or a fragment thereof, wherein the link may be either directly or through other polynucleotides of any sequence and of a length of about 1,000 base pairs, or about 500 base pairs, or about 300 base pairs, or about 200 base pairs, or about 150 base pairs, or about 100 base pairs or about 50 base pairs, or less.

The invention also particularly includes polynucleotide molecules, including DNA, that hybridize to, and are therefore the complements of, the nucleotide sequences of the disclosed GTSs. Such hybridization conditions may be highly stringent or less highly stringent, as described above. In instances wherein the nucleic acid molecules are deoxyoligonucleotides (“DNA oligos”), highly stringent conditions may refer to, for example, washing in 6×SSC/0.05% sodium pyrophosphate at 37° C. (for oligos having 14-base DNA oligos), 48° C. (for 17-base DNA oligos), 55° C. (for 20-base DNA oligos), and 60° C. (for 23-base oligos). Similar conditions are contemplated for RNA oligos corresponding to a portion of the disclosed GTS sequences.

These nucleic acid molecules may encode or act as antisense molecules to polynucleotides comprising at least a portion of the sequences shown in SEQ ID NOS:9-1008 that are useful, for example, to regulate the expression of genes comprising a nucleotide sequence of any of SEQ ID NOS:9-1008, and can also be used, for example, as antisense primers in amplification reactions of gene sequences. With respect to gene regulation, such techniques can be used to regulate, for example, developmental processes by modulating the expression of genes in embryonic stem cells. Further, such sequences may be used as part of ribozyme and/or triple helix sequences that can be used to regulate gene expression. Still further, such molecules may be used as components of diagnostic methods whereby, for example, the presence of a particular allele, of a gene that contains any of the sequences of SEQ ID NOS:9-1008 may be detected. Of particular interest is the use of the disclosed GTSs to conduct analysis of single nucleotide polymorphisms (SNPs), and particularly coding region SNPs or “cSNPs”, in the human genome, or as general or individual-specific forensic markers. When so applied, a collection of GTSs is obtained from an individual, and screened against a control database of cSNPs (or other genetic markers) that have previously been associated with disease, suitability or susceptibility (or sensitivity) to specific drugs or therapies, or virtually any other human trait that correlates with a given cSNP or genetic marker, or assortment thereof. In addition to disease/diagnostic testing, the described GTSs are also useful as genetic markers for the prenatal analysis of congenital traits or defects.

In addition to the nucleotide sequences described above, full length cDNA or gene sequences that contain any of SEQ ID NOS:9-1008 present in the same species and/or homologs of any of those genes present in other species can be identified and isolated by using molecular biological techniques known in the art.

In order to clone the full length cDNA sequence from any species encoding the cDNA corresponding to the entire messenger RNA or to clone variant or heterologous forms of the molecule, labeled DNA probes made from nucleic acid fragments corresponding to any of the partial cDNA disclosed herein may be used to screen a cDNA library. For example, oligonucleotides corresponding to either the 5′ or 3′ terminus of the cDNA sequence may be used to obtain longer nucleotide sequences. Briefly, the library may be plated out to yield a maximum of about 30,000 pfu for each 150 mm plate. Approximately 40 plates may be screened. The plates are incubated at 37° C. until the plaques reach a diameter of 0.25 mm or are just beginning to make contact with one another (3-8 hours). Nylon filters are placed onto the soft top agarose and after 60 seconds, the filters are peeled off and floated on a DNA denaturing solution consisting of 0.4N sodium hydroxide. The filters are then immersed in neutralizing solution consisting of 1 M Tris HCl, pH 7.5, before being allowed to air dry. The filters are prehybridized in casein hybridization buffer containing 10% dextran sulfate, 0.5 M NaCl, 50 mM Tris HCL, pH 7.5, 0.1% sodium pyrophosphate, 1% casein, 1% SDS, and denatured salmon sperm DNA at 0.5 mg/ml for 6 hours at 60° C. The radiolabelled probe is then denatured by heating to 95° C. for 2 minutes and then added to the prehybridization solution containing the filters. The filters are hybridized at 60° C. (alternatively, as in all hybridizations described herein, approximately 42, 44,46, 48, 50. 52, 54, 56, 58, 62, 64, 66, 68, 70, or about 72 degrees or more can be used) for about 16 hours. The filters are then washed in approximately 1× wash mix (10× wash mix contains 3M NaCl, 0.6M Tris base, and 0.02M EDTA, alternatively, as with all washes described herein, 2×, 3×, 4×, 5×, 6× wash mix, or more, can be used) twice for 5 minutes each at room temperature, then in 1× wash mix containing 1% SDS at 60° C. (alternatively, as in all washes described herein, approximately 42, 44, 46, 48, 50. 52, 54, 56, 58, 62, 64, 66, 68, 70, or about 72 degrees or more can be used) for about 30 min, and finally in 0.3× wash mix (alternatively, as in all final washes described herein, approximately, 0.2×, 0.4×, 0.6×, 0.8×, 1×, or any concentration between about 2× and about 6× can be used in conjunction with a suitable wash temperature) containing 0.1% SDS at 60° C. (alternatively, approximately 42, 44, 46, 48, 50. 52, 54, 56, 58, 62, 64, 66, 68, 70, or about 72 degrees or more can be used) for about 30 min. The filters are then air dried and exposed to x-ray film for autoradiography. After developing, the film is aligned with the filters to select a positive plaque. If a single, isolated positive plaque cannot be obtained, the agar plug containing the plaques will be removed and placed in lambda dilution buffer containing 0.1M NaCl, 0.01M magnesium sulfate, 0.035M Tris HCl, pH 7.5, 0.01% gelatin. The phage may then be replated and rescreened to obtain single, well isolated positive plaques. Positive plaques may be isolated and the cDNA clones sequenced using primers based on the known cDNA sequence. This step may be repeated until a full length cDNA is obtained.

It may be necessary to screen multiple cDNA libraries from different sources/tissues to obtain a full length cDNA. In the event that it is difficult to identify cDNA clones encoding the complete 5′ terminal coding region, an often encountered situation in cDNA cloning, the RACE (Rapid Amplification of cDNA Ends) technique may be used. RACE is a proven PCR-based strategy for amplifying the 5′ end of incomplete cDNAs. 5′-RACE-Ready cDNA synthesized from human fetal liver containing a unique anchor sequence is commercially available (Clontech). To obtain the 5′ end of the cDNA, PCR is carried out, for example, on 5′-RACE-Ready cDNA using the provided anchor primer and the 3′ primer. A secondary PCR reaction is then carried out using the anchored primer and a nested 3′ primer according to the manufacturer's instructions.

Once obtained, the full length cDNA sequence may be translated into amino acid sequence and examined for certain landmarks found in the amino acid sequences encoded by SEQ ID NOS:9-1008, or any structural similarities to these disclosed sequences.

The identification of homologs, heterologs, or paralogs of SEQ ID NOS:9-1008 in other, preferably related, species can be useful for developing additional animal model systems that are closely related to humans for purposes of drug discovery. Genes at other genetic loci within the genome that encode proteins which have extensive homology to one or more domains of the gene products encoded by SEQ ID NOS:9-1008 can also be identified via similar techniques. In the case of cDNA libraries, such screening techniques can identify clones derived from alternatively spliced transcripts in the same or different species.

Screening can be done using filter hybridization with duplicate filters. The labeled probe can contain at least 15-30 base pairs of the nucleotide sequence presented in SEQ ID NOS:9-1008. The hybridization washing conditions used should be of a lower stringency when the cDNA library is derived from an organism different from, or heterologous to, the type of organism from which the labeled sequence was derived. With respect to the cloning of a mammalian homolog, heterolog, ortholog, or paralog, using probes derived from any of the sequences of SEQ ID NOS:9-1008, for example, hybridization can, for example, be performed at 65° C. overnight in Church's buffer (7% SDS, 250 mM NaHPO₄, 2 mM EDTA, 1% BSA). Washes can be done with 2×SSC,0.1% SDS at 65° C. and then at 0.1×SSC,0.1% SDS at 65° C.

Low stringency conditions are well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

Alternatively, the labeled nucleotide probe of a sequence of any of SEQ ID NOS:9-1008 may be used to screen a genomic library derived from the organism of interest, again, using appropriately stringent conditions. The identification and characterization of human genomic clones is helpful for designing diagnostic tests and clinical protocols for treating disorders in human patients that are known or suspected to be linked to disease or other developmental or cell differentiation disorders and abnormalities. For example, sequences derived from regions adjacent to the intron/exon boundaries of the human gene can be used to design primers for use in amplification assays to detect mutations within the exons, introns, splice sites (e.g., splice acceptor and/or donor sites), etc., that can be used in diagnostics.

Further, gene homologs can also be isolated from nucleic acid of the organism of interest by performing PCR using two oligonucleotide primers derived from SEQ ID NOS:9-1008 or two degenerate oligonucleotide primer pools designed on the basis of amino acid sequences within the gene products encoded by SEQ ID NOS:9-1008. The template for the reaction may be cDNA obtained by reverse transcription of mRNA prepared from, for example, human or non-human cell lines, cell types, or tissues, like, for example, ES cells from the organism of interest.

The PCR product may be subcloned or sequenced directly or subcloned and sequenced to ensure that the amplified sequences represent the sequences of the gene corresponding to the sequence of SEQ ID NOS:9-1008 of interest. The PCR fragment may then be used to isolate a full length cDNA clone by a variety of methods. For example, the amplified fragment may be labeled and used to screen a cDNA library, such as a bacteriophage cDNA library. Alternatively, the labeled fragment may be used to isolate genomic clones via the screening of a genomic library.

PCR technology may also be utilized to isolate full length cDNA sequences. For example, RNA can be isolated using standard procedures from an appropriate cellular source (i.e., one known, or suspected, to express the gene corresponding to the sequence of SEQ ID NOS:9-1008 of interest, such as, for example, ES cells). A reverse transcription reaction may be performed on the RNA using an oligonucleotide primer specific for the most 5′ end of the amplified fragment for the priming of first strand synthesis. The resulting RNA/DNA hybrid may then be “tailed” with guanines, for example, using a standard terminal transferase reaction, the hybrid may be digested with RNase H, and second strand synthesis may then be primed with a poly-C primer. Thus, cDNA sequences upstream from the amplified fragment may easily be isolated. For a review of cloning strategies which may be used, see e.g., Sambrook et al., 1989, supra. Alternatively, cDNA or genomic libraries can be screened using 5′ PCR primers that hybridize to vector sequences and 3′ PCR primers specific to the gene of interest. Typically, such primers comprise oligonucleotide “priming” sequences first disclosed in, or otherwise unique to, one of the GTSs of SEQ ID NOS:9-1008.

The sequence of a gene corresponding to any of the sequences of SEQ ID NOS:9-1008 can also be used to isolate mutant alleles of that gene. Such mutant alleles may be isolated from individuals either known or suspected to have a genotype which contributes to the disease of interest or other symptoms of developmental and cell differentiation and/or proliferation disorders and abnormalities. Mutant alleles and mutant allele products may then be utilized in the therapeutic and diagnostic programs described below. Additionally, such sequences of any of the genes corresponding to SEQ ID NOS:9-1008 can be used to detect gene regulatory (e.g., promoter or promoter/enchancer) defects which can affect development or cell differentiation.

A cDNA of a mutant gene corresponding to any of the sequences of SEQ ID NOS:9-1008 can be isolated as discussed above, or, for example, by using PCR. In this case, the first cDNA strand may be synthesized by hybridizing an oligo-dT oligonucleotide to mRNA isolated from cells derived from an individual suspected of carrying a mutant gene corresponding to any of the sequences of SEQ ID NOS:9-1008 by extending the new strand with reverse transcriptase. The second strand of the cDNA is then synthesized using an oligonucleotide that hybridizes specifically to the 5′ region of the normal gene. The amplified product can be directly sequenced or cloned into a suitable vector and subsequently subjected to DNA sequence analysis. By comparing the DNA sequence of the mutant allele to that of the normal allele, the mutation(s) responsible for the loss or alteration of function of the mutant gene product can be ascertained.

Alternatively, a genomic library can be constructed using DNA obtained from one or more individuals suspected of carrying, or known to carry, a mutant allele corresponding to any of SEQ ID NOS:9-1008. Corresponding mutant cDNA libraries can be also constructed using RNA from cell types known, or suspected, to express such mutant alleles. The corresponding normal gene, or any suitable fragment thereof, may then be labeled and used as a probe to identify the corresponding mutant allele in such libraries. Clones containing the mutant gene sequences may then be identified and analyzed by DNA sequence analysis. Additionally, a protein expression library can be constructed utilizing cDNA synthesized from, for example, RNA isolated from a cell type known, or suspected, to express a mutant allele corresponding to any of the sequences of SEQ ID NOS:9-1008 from an individual suspected of, carrying or known to carry, such a mutant allele. In this manner, gene products made by the putatively mutant cell type may be expressed and screened using standard antibody screening techniques in conjunction with antibodies raised against the corresponding normal gene product or a portion thereof, as described below in Section 5.4 (For screening techniques, see, for example, Harlow, E. and Lane, eds., 1988, “Antibodies: A Laboratory Manual”, Cold Spring Harbor Press, Cold Spring Harbor.) Additionally, screening can be accomplished by screening with labeled fusion proteins. In cases where a mutation results in an expressed gene product with altered function (e.g., as a result of a missense or a frame shift mutation), a polyclonal set of antibodies to the wild-type gene product are likely to cross-react with the mutant gene product. Library clones detected via their reaction with such labeled antibodies can be purified and subjected to sequence analysis according to methods well known to those of skill in the art.

The invention also encompasses nucleotide sequences that encode mutant isoforms of any of the amino acid sequences encoded by the GTSs of SEQ ID NOS:9-1008, peptide fragments thereof, truncated versions thereof, and fusion proteins including any of the above. Examples of such fusion proteins can include, but not limited to, an epitope tag which aids in purification or detection of the resulting fusion protein; or an enzyme, fluorescent protein, luminescent protein which can be used as a marker.

The present invention additionally encompasses (a) RNA or DNA vectors that contain any portion of SEQ ID NOS:9-1008 and/or their complements as well as any of the peptides or proteins encoded thereby; (b) DNA vectors that contain a cDNA that substantially spans the entire open reading frame corresponding to any of the sequences of SEQ ID NOS:9-1008 and/or their complements; (c) DNA expression vectors that have or contain any of the foregoing sequences, or a portion thereof, operatively associated with a (d) genetically engineered host cells that contain a cDNA that spans the entire open reading frame, or any portion thereof, corresponding to any of the sequences of SEQ ID NOS:9-1008 operatively associated with a regulatory element, generally recombinantly positioned either in vivo (such as in gene activation) or in vitro that directs the expression of the coding sequences in the host cell. As used herein, regulatory elements include, but are not limited to, inducible and non-inducible promoters, enhancers, operators and other elements known to those skilled in the art that drive and regulate expression. Such regulatory elements include, but are not limited to, the baculovirus promoter, cytomegalovirus hCMV immediate early gene promoter, the early or late promoters of SV40 adenovirus, the lac system, the trp system, the TAC system, the TRC system, the major operator and promoter regions of phage A, the control regions of fd coat protein, acid phosphatase promoters, phosphoglycerate kinase (PGK) and especially 3-phosphoglycerate kinase promoters, and yeast alpha mating factors.

An additional application of the described novel human polynucleotide sequences is their use in the molecular mutagenesis/evolution of proteins that are at least partially encoded by the described novel sequences using, for example, polynucleotide shuffling or related methodologies. Such approaches are described in U.S. Pat. Nos. 5,830,721 and 5,837,458 hich are herein incorporated by reference in their entirety.

5.2 Proteins and Polypeptides Encoded by Polynucleotides Expressed in Modified Human Cells

Peptides and proteins encoded by the open reading frame of mRNAs corresponding to SEQ ID NOS:9-1008, polypeptides and peptide fragments, mutated, truncated or deleted forms of those peptides and proteins, fusion proteins containing any of those peptides and proteins can be prepared for a variety of uses, including, but not limited to, the generation of antibodies, as reagents in diagnostic assays, the identification of other cellular gene products involved in the regulation of development and cellular differentiation of various cell types, like, for example, ES cells, as reagents in assays for screening for compounds that can be used in the treatment of disorders affecting development and cell differentiation, and as pharmaceutical reagents useful in the treatment of disorders affecting development and cell differentiation.

The invention also encompasses proteins, peptides, and polypeptides that are functionally equivalent to those encoded by SEQ ID NOS:9-1008. Such functionally equivalent products include, but are not limited to, additions or substitutions of amino acid residues within the amino acid sequence encoded by the nucleotide sequences described above, but which result in a silent change, thus producing a functionally equivalent gene product. Amino acid substitutions can be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

While random mutations can be introduced into DNA encoding peptides and proteins of the current invention (using random mutagenesis techniques well known to those skilled in the art), and the resulting mutant peptides and proteins tested for activity, site-directed mutations of the coding sequence can be engineered (using standard site-directed mutagenesis techniques) to generate mutant peptides and proteins of the current invention having increased functionality.

For example, the amino acid sequence of peptides and proteins of the current invention can be aligned with homologs from different species. Mutant peptides and proteins can be engineered so that regions of interspecies identity are maintained, whereas the variable residues are altered, e.g., by deletion or insertion of an amino acid residue(s) or by substitution of one or more different amino acid residues. Conservative alterations at the variable positions can be engineered in order to produce a mutant form of a peptide or protein of the current invention that retains function. Non-conservative changes can be engineered at these variable positions to alter function. Alternatively, where alteration of function is desired, deletion or non-conservative alterations of the conserved regions can be engineered. One of skill in the art may easily test such mutant or deleted form of a peptide or protein of the current invention for these alterations in function using the teachings presented herein.

Other mutations to the coding sequences described above can be made to generate peptides and proteins that are better suited for expression, scale up, etc. in the host cells chosen. For example, the triplet code for each amino acid can be modified to conform more closely to the preferential codon usage of the host cell's translational machinery, or, for example, to yield a messenger RNA molecule with a longer half-life. Those skilled in the art would readily know what modifications of the nucleotide sequence would be desirable to conform the nucleotide sequence to preferential codon usage or to make the messenger RNA more stable. Such information would be obtainable, for example, through use of computer programs, through review of available research data on codon usage and messenger RNA stability, and through other means known to those of skill in the art.

Peptides corresponding to one or more domains (or a portion of a domain) of one of the proteins described above, truncated or deleted proteins, as well as fusion proteins in which the full length protein described above, a subunit peptide or truncated version is fused to an unrelated protein are also within the scope of the invention and can be designed by those of skill in the art on the basis of experimental or functional considerations. Such fusion proteins include, but are not limited to, fusions to an epitope tag; or fusions to an enzyme, fluorescent protein, or luminescent protein which provide a marker function.

While the peptides and proteins of the current invention can be chemically synthesized (e.g., see Creighton, 1983, Proteins: Structures and Molecular Principles, W.H. Freeman & Co., N.Y.), large polypeptides derived from any of the polynucleotides described above may advantageously be produced by recombinant DNA technology using techniques well known in the art for expressing genes and/or coding sequences. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding any of the nucleotide sequences described above may be chemically synthesized using, for example, synthesizers. See, for example, the techniques described in “Oligonucleotide Synthesis”, 1984, Gait, M. J. ed., IRL Press, Oxford, which is incorporated by reference herein in its entirety.

A variety of host-expression vector systems may be utilized to express the nucleotide sequences of the invention. Where the peptide or protein to be synthesized is a soluble derivative, the peptide or polypeptide can be recovered from the culture, i.e., from the host cell in cases where the peptide or polypeptide is not secreted, and from the culture media in cases where the peptide or polypeptide is secreted by the cells. However, such engineered host cells themselves may be used in situations where it is important not only to retain the structural and functional characteristics of the expressed peptide or protein, but to assess biological activity, e.g., in drug screening assays.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors containing a nucleotide sequence of the current invention; yeast (e.g., Saccharomyces, Pichia) transformed with recombinant yeast expression vectors containing a nucleotide sequence of the current invention; insect cell systems infected with recombinant virus expression vectors (e.g., baculovirus) containing a nucleotide sequence of the current invention; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a nucleotide sequence of the current invention; or mammalian cell systems (e.g., COS, CHO, BHK, 293, 3T3, U937) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the gene product being expressed. For example, when large quantities of such a protein are to be produced for the generation of pharmaceutical compositions of a protein or for raising antibodies to the protein to be expressed, for example, vectors which direct the expression of high levels of fusion protein products that are readily purified may be desirable. Such vectors include, but are not limited to, the E. coli expression vector pUR278 (Ruther et al., 1983, EMBO J. 2:1791), in which the coding sequence of the polynucleotide to be expressed may be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). If the inserted sequence encodes a relatively small polypeptide (less than 25 kD), such fusion proteins are generally soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety. Alternatively, if the resulting fusion protein is insoluble and forms inclusion bodies in the host cell, the inclusion bodies may be purified and the recombinant protein solubilized using techniques well known to one of skill in the art.

In an insect system, Autographa californica nuclear polyhidrosis virus (AcNPV) may be used as a vector to express foreign genes. (e.g., see Smith et al., 1983, J. Virol. 46: 584; Smith, U.S. Pat. No. 4,215,051). In one embodiment of the current invention, Sf9 insect cells are infected with a baculovirus vector expressing a peptide or protein of the current invention.

In mammalian host cells, a number of viral-based expression systems may be utilized. Specific embodiments (described more fully below) include the gene trap cDNA sequences of the current invention that are expressed by a CMV promoter to transiently express recombinant protein in U937 cells or in Cos-7 cells. Alternatively, retroviral vector systems well known in the art may be used to insert the recombinant expression construct into host cells, or vaccinia virus-based expression systems may be employed.

In yeast, a number of vectors containing constitutive or inducible promoters may be used. For a review, see Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol.153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathem et al., Cold Spring Harbor Press, Vols. I and II.

In cases where plant expression vectors are used, the expression of the coding sequence may be driven by any of a number of promoters. For example, viral promoters such as the 35S RNA and 19S RNA promoters of CaMV (Brisson et al, 1984, Nature, 310:511-514), or the coat protein promoter of TMV (Takamatsu et al., 1987, EMBO J. 6:307-311) may be used; alternatively, plant promoters such as the small subunit of RUBISCO (Coruzzi et al., 1984, EMBO J. 3:1671-1680; Broglie et al., 1984, Science 224:838-843); or heat shock promoters, e.g., soybean hsp17.5-E or hsp17.3-B (Gurley et al., 1986, Mol. Cell. Biol. 6:559-565) may be used. These constructs can be introduced into plant cells using Ti plasmids, Ri plasmids, plant virus vectors, direct DNA transformation, microinjection, electroporation, etc. For reviews of such techniques see, for example, Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.

In cases where an adenovirus is used as an expression vector, the nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the gene product of interest in infected hosts. (e.g., See Logan & Shenk, 1984, Proc. Natl. Acad. Sci. USA 81:3655-3659). Specific initiation signals may also be required for efficient translation of inserted nucleotide sequences of interest. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of a coding sequence of interest is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enchanter elements, transcription terminators, etc. (See Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript may be used. Such mammalian host cells include, but are not limited to, CHO, VERO, BHK, HeLa, COS, MDCK, 293, 3T3, WI38, and U937 cells.

For long-term, high-yield production of recombinant proteins, stable expression is preferred. For example, cell lines which stably express the sequences of interest described above may be engineered. Rather than using expression vectors which contain viral origins of replication, host cells can be transformed with DNA controlled by appropriate expression control elements (e.g., promoter, enchanter sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the gene product of interest. Such engineered cell lines may be particularly useful in screening and evaluation of compounds that affect the endogenous activity of the gene product of interest.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for the following genes: dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147).

The gene products of interest can also be expressed in transgenic animals. Animals of any species, including, but not limited to, mice, rats, rabbits, guinea pigs, pigs, micro-pigs, goats, and non-human primates, e.g., baboons, monkeys, and chimpanzees may be used to generate transgenic animals carrying the polynucleotide of interest of the current invention.

Any technique known in the art may be used to introduce the transgene of interest into animals to produce the founder lines of transgenic animals. Such techniques include, but are not limited to pronuclear microinjection (Hoppe, P. C. and Wagner, T. E., 1989, U.S. Pat. No. 4,873,191); retrovirus mediated gene transfer into germ lines (Van der Putten et al., 1985, Proc. Natl. Acad. Sci., USA 82:6148-6152); gene targeting in embryonic stem cells (Thompson et al., 1989, Cell 56:313-321); electroporation of embryos (Lo, 1983, Mol Cell. Biol. 3:1803-1814); sperm-mediated gene transfer (Lavitrano et al., 1989, Cell 57:717-723); positive-negative selection as described in U.S. Pat. No. 5,464,764 herein incorporated by reference. For a review of such techniques, see Gordon, 1989, Transgenic Animals, Intl. Rev. Cytol. 115:171-229, which is incorporated by reference herein in its entirety.

The present invention provides for transgenic animals that carry the transgene of interest in all their cells, as well as animals which carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, M. et al., 1992, Proc. Natl. Acad. Sci. USA 89:6232-6236). The regulatory sequences required for such a cell-type specific activation will depend upon the particular cell type of interest, and will be apparent to those of skill in the art. When it is desired that the transgene of interest be integrated into the chromosomal site of the endogenous copy of that same gene, gene targeting is preferred. Briefly, when such a technique is to be utilized, vectors containing some nucleotide sequences homologous to the endogenous gene of interest are designed for the purpose of integrating, via homologous recombination with chromosomal sequences, into and disrupting the function of the nucleotide sequence of the endogenous gene of interest. In this way, the expression of the endogenous gene may also be eliminated by inserting non-functional sequences into the endogenous gene. The transgene may also be selectively introduced into a particular cell type, thus inactivating the endogenous gene of interest in only that cell type, by following, for example, the teaching of Gu et al. (Gu et al., 1994, Science 265: 103-106). The regulatory sequences required for such a cell-type specific inactivation will depend upon the particular cell type of interest and will be apparent to those of skill in the art.

Once transgenic animals have been generated, the expression of the recombinant gene of interest may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include, but are not limited to, Northern blot analysis of cell type samples obtained from the animal, in situ hybridization analysis, and RT-PCR. Samples of gene-expressing tissue, may also be evaluated immunocytochemically using antibodies specific for the transgene product, as described below.

5.3 Cells that Contain a Disrupted Allele of a Gene Encoding a Polynucleotide of the Current Invention

Another aspect of the current invention are cells which contain a gene that encodes a polynucleotide of the current invention and that has been disrupted. Those of skill in the art would know how to disrupt a gene in a cell using techniques known in the art. Also, techniques useful to disrupt a gene in a cell and especially an ES cell, that may already be disrupted, as disclosed in copending U.S. patent application Ser. Nos. 08/726,867; 08/728,963; 08/907,598; and 08/942,806, all of which are hereby incorporated herein by reference in their entirety, are within the scope of the current invention to disrupt a gene that encodes a polynucleotide of the current invention.

5.3.1 Identification of Cells that Express Genes Encoding Polynucleotides of the Current Invention

Host cells that contain coding sequence and/or express a biologically active gene product, or fragment thereof, encoded by a gene corresponding to a GTS present invention may be identified by at least four general approaches; (a) DNA-DNA or DNA-RNA hybridization; (b) the presence or absence of “marker” gene functions; (c) assessing the level of transcription as measured by the expression of mRNA transcripts in the host cell; and (d) detection of the gene product as measured by immunoassay, enzymatic assay, chemical assay, or by its biological activity. Prior to screening for gene expression, the host cells can first be treated in an effort to increase the level of expression of genes encoding polynucleotides of the current invention, especially in cell lines that produce low amounts of the mRNAs and/or peptides and proteins of the current invention.

In the first approach, the presence of the coding sequence for peptides and proteins of the current invention inserted in the expression vector can be detected by DNA-DNA or DNA-RNA hybridization using probes comprising nucleotide sequences that are homologous to the coding sequence for peptides and proteins of the current invention, respectively, or portions or derivatives thereof.

In the second approach, the recombinant expression vector/host system can be identified and selected based upon the presence or absence of certain “marker” gene functions (e.g., thymidine kinase activity, resistance to antibiotics, resistance to methotrexate, transformation phenotype, occlusion body formation in baculovirus, etc.). For example, if the coding sequence for the peptide or protein of the current invention is inserted within a marker gene sequence of the vector, recombinants containing the coding sequence for the peptide or protein of the current invention can be identified by the absence of the marker gene function. Alternatively, a marker gene can be placed in tandem with the sequence for the peptide or protein of the current invention under the control of the same or different promoter used to control the expression of the coding sequence for the peptide or protein of the current invention. Expression of the marker in response to induction or selection indicates expression of the coding sequence for the peptide or protein of the current invention.

In the third approach, transcriptional activity for the coding region of genes specific for peptides and proteins of the current invention can be assessed by hybridization assays. For example, RNA can be isolated and analyzed by Northern blot using a probe derived from a GTS, or any portion thereof. Alternatively, total nucleic acids of the host cell may be extracted and assayed for hybridization to such probes. Additionally, RT-PCR (using GTS specific oligos/products) may be used to detect low levels of gene expression in a sample, or in RNA isolated from a spectrum of different tissues, or PCR can be used can be used to screen a variety of cDNA libraries derived from different tissues to determine which tissues express a given GTS.

In the fourth approach, the expression of the peptides and proteins of the current invention can be assessed immunologically, for example by Western blots, immunoassays such as radioimmuno-precipitation, enzyme-linked immunoassays and the like. This can be achieved by using an antibody and a binding partner specific to a peptide or protein of the current invention.

5.4 Antibodies to Proteins of the Current Invention

Antibodies that specifically recognize one or more epitopes of a peptide or protein of the current invention, or epitopes of conserved variants of a peptide or protein at least partially encoded by a GTS of the present invention, or any and all peptide fragments thereof, are also encompassed by the invention. Such antibodies include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in the detection of the peptide or protein of interest of the current invention in a biological sample and may, therefore, be utilized as part of a diagnostic or prognostic technique whereby patients may be tested for abnormal amounts of these proteins. Such antibodies may also be utilized in conjunction with, for example, compound screening schemes as described, below in Section 5.6 for the evaluation of the effect of test compounds on expression and/or activity of the gene products of interest of the current invention. Additionally, such antibodies can be used in conjunction with the gene therapy and gene delivery techniques described below to, for example, evaluate the normal and/or engineered peptide- or protein-expressing cells prior to their introduction into the patient. Such antibodies may additionally be used as a method for inhibiting the abnormal activity of a peptide or protein of interest at least partially encoded by a GTS of the present invention. Thus, such antibodies may, for example, be utilized as part of treatment methods for development and cell differentiation disorders.

For the production of antibodies, various host animals may be immunized by injection with the peptide or protein of interest, a subunit peptide of such protein, a truncated polypeptide, functional equivalents of the peptide or protein, mutants of the peptide or protein, or denatured forms of the above. Such host animals may include, but are not limited to, rabbits, mice, and rats, to name but a few. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjutants such as BCG (bacille Calmette-Guerin) and Corynebacterium parvum. Polyclonal antibodies are heterogeneous populations of antibody molecules derived from the sera of the immunized animals.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci. USA, 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a porcine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce single chain antibodies against gene products of interest. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include, but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Antibodies to peptides and proteins that are fully or at least partially encoded by the described GTSs, or fragments or truncated versions thereof, can in turn be utilized to generate anti-idiotypic antibodies that “mimic” an epitope of the peptide or protein of interest, using techniques well known to those skilled in the art. (See, e.g., Greenspan & Bona, 1993, FASEB J 7(5):437-444; and Nissinoff, 1991, J. Immunol. 147(8):2429-2438). For example antibodies that bind to a regulatory peptide or protein of interest of the current invention and competitively inhibit the binding of such peptide or protein to any of its binding partners in the cell can be used to generate anti-idiotypes that “mimic” the peptide or protein of interest and, therefore, bind and neutralize the particular binding partner of the peptide or protein of interest. Such neutralizing antibodies, anti-idiotypes, Fab fragments of such antibodies, or humanized derivatives thereof, can be used in therapeutic regimens to mimic or neutralize (depending on the antibody) the effect of a particular peptide of interest, or a binding partner of a peptide or protein of interest.

5.5 Diagnosis of Disorders Affecting Development and Cell Differentiation

A variety of methods can be employed for the diagnostic and prognostic evaluation of disorders involving developmental and differentiation processes, and for the identification of subjects having a predisposition to such disorders.

Such methods may, for example, utilize reagents such as the nucleotide sequences described above, and antibodies to peptides and proteins of the current invention, as described, in Section 5.4. Specifically, such reagents may be used, for example, for: (1) the detection of the presence of gene mutations, or the detection of either over- or under-expression of the respective mRNAs relative to the non-disorder state; (2) the detection of either an over- or an under-abundance of the respective gene product relative to the non-disorder state; and (3) the detection of perturbations or abnormalities in the intra- and inter-cellular processes mediated by the respective peptides or proteins of the current invention.

The methods described herein may be performed, for example, by utilizing pre-packaged diagnostic kits comprising at least one specific nucleotide sequence of the current invention or antibody reagent described herein, which may be conveniently used, e.g., in clinical settings, to diagnose patients exhibiting developmental or cell differentiation disorder abnormalities.

For the detection of mutations in any of the genes described above, any nucleated cell can be used as a starting source for genomic nucleic acid. For the detection of gene expression or gene products, any cell type or tissue in which the gene of interest is expressed, such as, for example, ES cells, may be utilized. Specific examples of cells and tissues that can be analyzed using the claimed polynucleotides include, but are not limited to, endothelial cells, epithelial cells, islets, neurons or neural tissue, mesothelial cells, osteocytes, lymphocytes, chondrocytes, hematopoietic cells, immune cells, cells of the major glands or organs (e.g., lung, heart, stomach, pancreas, kidney, skin, etc.), exocrine and/or endocrine cells, embryonic and other stem cells, fibroblasts, and culture adapted and/or transformed versions of the above. Diseases or natural processes that can also be correlated with the expression of mutant, or normal, variants of the disclosed GTSs include, but are not limited to, aging, cancer, autoimmune disease, lupus, scleroderma, Crohn's disease, multiple sclerosis, inflammatory bowel disease, immune disorders, schizophrenia, psychosis, alopecia, glandular disorders, inflammatory disorders, ataxia telangiectasia, diabetes, skin disorders such as acne, eczema, and the like, osteo and rheumatoid arthritis, high blood pressure, atherosclerosis, cardiovascular disease, pulmonary disease, degenerative diseases of the neural or skeletal systems, Alzheimer's disease, Parkinson's disease, osteoporosis, asthma, developmental disorders or abnormalities, genetic birth defects, infertility, epithelial ulcerations, and viral, parasitic, fungal, yeast, or bacterial infection.

Primary, secondary, or culture-adapted variants of cancer cells/tissues can also be analyzed using the claimed polynucleotides. Examples of such cancers include, but are not limited to, Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiforme, oligodendroglioma, schwannoma, retinoblastoma, congenital tumors), spinal cord (neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, endometrioid tumors, celioblastoma, clear cell carcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma [embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles, dysplastic nevi, lipoma, angioma, dermatofibroma, keloids, psoriasis; Brcast: carcinoma and sarcoma, and Adrenal glands: neuroblastoma.

Nucleic acid-based detection techniques and peptide detection techniques that can be used to conduct the above analyses are described below.

5.5.1. Detection of the Genes of the Current Invention and Their Respective Transcripts

Mutations within the genes of the current invention can be detected by utilizing a number of techniques. Nucleic acid from any nucleated cell can be used as the starting point for such assay techniques, and may be isolated according to standard nucleic acid preparation procedures which are well known to those of skill in the art.

DNA may be used in hybridization or amplification assays of biological samples to detect abnormalities involving gene structure, including point mutations, insertions, deletions and chromosomal rearrangements. Such assays may include, but are not limited to, Southern analyses, single stranded conformational polymorphism analyses (SSCP), and PCR analyses.

Such diagnostic methods for the detection of gene-specific mutations can involve for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including recombinant DNA molecules, cloned genes or degenerate variants thereof, as described above, under conditions favorable for the specific annealing of these reagents to their complementary sequences within the gene of interest of the current invention. Preferably, the lengths of these nucleic acid reagents are at least 15 to 30 nucleotides. After incubation, all non-annealed nucleic acids are removed from the nucleic acid molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid reagents of the type described above are easily removed. Detection of the remaining, annealed, labeled nucleic acid reagents is accomplished using standard techniques well-known to those in the art. The gene sequences to which the nucleic acid reagents have annealed can be compared to the annealing pattern expected from a normal gene sequence in order to determine whether a gene mutation is present.

Alternative diagnostic methods for the detection of gene specific nucleic acid molecules, in patient samples or other appropriate cell sources, may involve their amplification, e.g., by PCR (the experimental embodiment set forth in Mullis, K. B., 1987, U.S. Pat. No. 4,683,202), followed by the detection of the amplified molecules using techniques well known to those of skill in the art. The resulting amplified sequences can be compared to those which would be expected if the nucleic acid being amplified contained only normal copies of the respective gene in order to determine whether a gene mutation exists.

Additionally, well-known genotyping techniques can be performed to identify individuals carrying mutations in any of the genes of the current invention. Such techniques include, for example, the use of restriction fragment length polymorphisms (RFLPs), which involve sequence variations in one of the recognition sites for the specific restriction enzyme used.

Furthermore, the polynucleotide sequences of the current invention may be mapped to chromosomes and specific regions of chromosomes using well known genetic and/or chromosomal mapping techniques. These techniques include in situ hybridization, linkage analysis against known chromosomal markers, hybridization screening with libraries or flow-sorted chromosomal preparations specific to known chromosomes, and the like. The technique of fluorescent in situ hybridization of chromosome spreads has been described, for example, in Verma et al. (1988) Human Chromosomes: A Manual of Basic Techniques, Pergamon Press, New York. Fluorescent in situ hybridization of chromosomal preparations and other physical chromosome mapping techniques may be correlated with additional genetic map data. Examples of genetic map data can be found, for example, in Genetic Maps: Locus Maps of Complex Genomes, Book 5: Human Maps, O'Brien, editor, Cold Spring Harbor Laboratory Press (1990). Comparisons of physical chromosomal map data may be of particular interest in detecting genetic diseases in carrier states.

The level of expression of genes can also be assayed by detecting and measuring the transcription of such genes. For example, RNA from a cell type or tissue known, or suspected to express any of the genes of the current invention can be isolated and tested utilizing hybridization or PCR techniques (e.g., northern or RT PCR) such as those described, above. Such analyses may reveal both quantitative and qualitative aspects of the expression pattern of the respective gene, including activation or inactivation of gene expression. In situ hybridization using suitable radioactive labels, enzymatic labels, or chemically tagged forms of the described polynucleotide sequences can also be used to assess expression patterns in vivo.

Additionally, an oligonucleotide or polynucleotide sequence first disclosed in at least a portion of one of the GTS sequences of SEQ ID NOS:9-1008 can be used as a hybridization probe in conjunction with a solid support matrix/substrate (resins, beads, membranes, plastics, polymers, metal or metallized substrates, crystalline or polycrystalline substrates, etc.). Of particular note are spatially addressable arrays (i e., gene chips, microtiter plates, etc.) of oligonucleotides and polynucleotides, or corresponding oligopeptides and polypeptides, wherein at least one of the biopolymers present on the spatially addressable array comprises an oligonucleotide or polynucleotide sequence first disclosed in at least one of the GTS sequences of SEQ ID NOS:9-1008, or an amino acid sequence encoded thereby. Methods for attaching biopolymers to, or synthesizing biopolymers on, solid support matrices, and conducting binding studies thereon are disclosed in, inter alia, U.S. Pat. Nos. 5,556,752, 5,744,305, 4,631,211, 5,445,934, 5,252,743, 4,713,326, 5,424,186, and 4,689,405 the disclosures of which are herein incorporated by reference in their entirety.

Oligonucleotides corresponding to the described GTSs can be used as hybridization probes either singly or in chip format. For example, a series of such GTS oligonucleotide sequences, or the complements thereof, can be used to represent all or a portion of the described GTS sequences. The oligonucleotides, typically between about 16 to about 40 (or any whole number within the stated range) nucleotides in length, may partially overlap each other and/or the NHP sequence may be represented using oligonucleotides that do not overlap. Accordingly, the described NHP polynucleotide sequences shall typically comprise at least about two or three distinct oligonucleotide sequences of at least about 18, and preferably about 25, nucleotides in length that are first disclosed in the described Sequence Listing. Such oligonucleotide sequences may begin at any nucleotide present within a sequence in the Sequence Listing and proceed in either a sense (5′-to-3′) orientation vis-a-vis the described sequence or in an antisense orientation.

Although the presently described GTSs have been specifically described using nucleotide sequence, it should be appreciated that each of the GTSs can uniquely be described using any of a wide variety of additional structural attributes, or combinations thereof. For example, a given GTS can be described by the net composition of the nucleotides present within a given region of the GTS in conjunction with the presence of one or more specific oligonucleotide sequence(s) first disclosed in the GTS. Alternatively, a restriction map specifying the relative positions of restriction endonuclease digestion sites, or various palindromic or other specific oligonucleotide sequences can be used to structurally describe a given GTS. Such restriction maps, which are typically generated by widely available computer programs (e.g., the University of Wisconsin GCG sequence analysis package, SEQUENCHER 3.0, Gene Codes Corp., Ann Arbor, Mich., etc.), can optionally be used in conjunction with one or more discrete nucleotide sequence(s) present in the GTS that can be described by the relative position of the sequence relative to one or more additional sequence(s) or one or more restriction sites present in the GTS.

5.5.2 Detection of the Gene Products of the Current Invention

Antibodies directed against wild type or mutant gene products of the current invention or conserved variants or peptide fragments thereof, which are discussed above in Section 5.4 may also be used as diagnostics and prognostics for disorders affecting development and cellular differentiation, as described herein. Such diagnostic methods, may be used to detect abnormalities in the level of gene expression, or abnormalities in the structure and/or temporal, tissue, cellular, or subcellular location of the respective gene product, and may be performed in vivo or in vitro, such as, for example, on biopsy tissue.

The tissue or cell type to be analyzed will generally include those which are known, or suspected, to contain cells that express the respective gene. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.), which is incorporated herein by reference in its entirety. The isolated cells can be derived from cell culture or from a patient. The analysis of cells taken from culture may be a necessary step in the assessment of cells that could be used as part of a cell-based gene therapy technique or, alternatively, to test the effect of compounds on the expression of the respective gene.

For example, antibodies, or fragments of antibodies, such as those described above in Section 5.4 are also useful in the present invention to quantitatively or qualitatively detect the presence of gene products of the current invention or conserved variants or peptide fragments thereof. This can be accomplished, for example, by immunofluorescence techniques employing a fluorescently labeled antibody (see below, this Section) coupled with light microscopic, flow cytometric, or fluorimetric detection.

The antibodies (or fragments thereof) or fusion or conjugated proteins useful in the present invention may, additionally, be employed histologically, as in immunofluorescence, immunoelectron microscopy or non-immuno assays, for in situ detection of gene products of the current invention or conserved variants or peptide fragments thereof, or for catalytic subunit binding (in the case of labeled catalytic subunit fusion protein).

In situ detection may be accomplished by removing a histological specimen from a patient, and applying thereto a labeled antibody or fusion protein of the present invention. The antibody (or fragment) or fusion protein is preferably applied by overlaying the labeled antibody (or fragment) onto a biological sample. Through the use of such a procedure, it is possible to determine not only the presence of the gene product of the current invention, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for gene products of the current invention or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying the respective gene products of interest or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled antibody specific to the peptide or protein of interest of the current invention or with fusion protein. The solid phase support may then be washed with the buffer a second time to remove unbound antibody or fusion protein. The amount of bound label on solid support may then be detected by conventional means.

“Solid phase support or carrier” is intended to encompass any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of antibody or fusion protein may be determined according to well known methods. Those skilled in the art will be able to determine operative and optimal assay conditions for each determination by employing routine experimentation.

With respect to antibodies, one of the ways in which the antibody can be detectably labeled is by linking the same to an enzyme and use in an enzyme immunoassay (EIA) (Voller, “The Enzyme Linked Immunosorbent Assay (ELISA)”, 1978, Diagnostic Horizons 2:1-7, Microbiological Associates Quarterly Publication, Walkersville, Md.); Voller et al., 1978, J. Clin. Pathol. 31:507-520; Butler, 1981, Meth. Enzymol. 73:482-523; Maggio (ed.), 1980, Enzyme Immunoassay, CRC Press, Boca Raton, Fla.,; Ishikawa et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect the peptide or protein of interest through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for labeling purposes include, but are not limited to, luciferin, luciferase and aequorin.

An additional use of a peptide or polypeptide encoded by an oligonucleotide or polynucleotide sequence first disclosed in at least one of the GTS sequences of SEQ ID NOS:9-1008 is by incorporating the sequence into a phage display, or other peptide library/binding, system that can be used to screen for proteins, or other ligands, that are capable of binding to an amino acid sequence encoded by an oligonucleotide or polynucleotide sequence first disclosed in at least one of the GTS sequences of SEQ ID NOS:9-1008 (see U.S. Pat. Nos. 5,270,170, and 5,432,018, herein incorporated by reference in their entirety). Moreover, peptide arrays comprising a novel amino acid sequence corresponding to a portion of at least one of the polynucleotide sequences first disclosed in SEQ ID NOS:9-1008 can be generated and screened essentially as described in U.S. Pat. Nos. 5,143,854, 5,405,783, and 5,252,743, the complete disclosures of which are herein incorporated by references.

Additionally, the presently described GTSs, or primers derived therefrom, can be used to screen spatially addressable arrays, or pools therefrom, of clones present in a full-length human cDNA library. The 96 well microtiter plate format is especially well-suited to the screening, by PCR for example, of pooled subfractions of cDNA clones.

5.6 Screening Assays for Compounds that Modulate the Expression or Activity of Peptides and Proteins of the Current Invention

The following assays are designed to identify compounds that interact with (e.g., bind to) peptides and proteins at least partially encoded by one of SEQ ID NOS:9-1008 (i.e. peptides or proteins of the current invention) compounds that interact with (e.g., bind to) intracellular proteins that interact with peptides and proteins of the current invention, compounds that interfere with the interaction of peptides and proteins of the current invention with each other and with other intracellular proteins involved in developmental and cell differentiation processes, and to compounds which modulate the activity of genes of the current invention (i.e., modulate the level of expression of genes of the current invention) or modulate the level of gene products of the current invention. Assays may additionally be utilized which identify compounds which bind to gene regulatory sequences (e.g., promoter sequences) and which may modulate the expression of genes of the current invention. See e.g., Platt, K. A., 1994, J. Biol. Chem. 269:28558-28562, which is incorporated herein by reference in its entirety.

Compounds that can be screened in accordance with the invention include, but are not limited to, peptides, antibodies and fragments thereof, prostaglandins, lipids and other organic compounds (e.g., terpines, peptidomimetics) that bind to the peptide or protein of interest of the current invention and either mimic the activity triggered by the natural ligand (i.e., agonists) or inhibit the activity triggered by the natural ligand (i.e., antagonists); as well as peptides, antibodies or fragments thereof, and other organic compounds that mimic the peptide or protein of interest of the current invention (or a portion thereof) and bind to and “neutralize” natural ligand.

Such compounds may include, but are not limited to, peptides such as, for example, soluble peptides, including but not limited to members of random peptide libraries (see, e.g., Lam, K. S. et al., 1991, Nature 354:82-84; Houghten, R. et al., 1991, Nature 354:84-86), and combinatorial chemistry-derived molecular library peptides made of D- and/or L-configuration amino acids, phosphopeptides (including, but not limited to, members of random or partially degenerate, directed phosphopeptide libraries; see, e.g., Songyang, Z. et al., 1993, Cell 72:767-778); antibodies (including, but not limited to, polyclonal, monoclonal, humanized, anti-idiotypic, chimeric or single chain antibodies, and Fab, F(ab′)₂ and Fab expression library fragments, and epitope-binding fragments thereof); and small organic or inorganic molecules.

Other compounds that can be screened in accordance with the invention include, but are not limited to, small organic molecules that are able to gain entry into an appropriate cell (e.g., in ES cells) and affect the expression of a gene of the current invention or some other gene involved in development and cell differentiation (e.g. by interacting with the regulatory region or transcription factors involved in gene expression); or such compounds that affect the activity of the peptide or protein of interest of the current invention, e.g., by inhibiting or enhancing the binding of such peptide or protein to another cellular peptide or protein, or other factor, necessary for catalysis, signal transduction, or the like, that is involved in developmental or cell differentiation processes.

Computer modeling and searching technologies permit the identification of compounds, or the improvement of already identified compounds, that can modulate the expression or activity of peptides or proteins of interest of the current invention. Having identified such a compound or composition, the active sites or regions are identified. Such active sites might typically be the binding partner sites, such as, for example, the interaction domains of the peptides and proteins of the current invention with their respective binding partners. The active site can be identified using methods known in the art including, for example, from study of the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential modulating compounds of the peptides and proteins of interest of the current invention.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner, systematic variations in composition, such as by varying side groups, can be quickly evaluated to obtain modified modulating compounds or ligands of improved specificity or activity.

Further experimental and computer modeling methods useful to identify modulating compounds based upon identification of the active sites of peptides and proteins of interest of the current invention, and related factors involved in development, cellular differentiation, and other cellular processes will be apparent to those of skill in the art.

Examples of molecular modeling systems are the CHARM and QUANTA programs (Polygon Corporation, Waltham, Mass.). CHARM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxicol. 29:111- 122; Perry and Davies, OSAR: Quantitative Structure-Activity Relationships in Drug Design pp. 189-193 (Alan R. Liss, Inc. 1989); Lewis and Dean, 1989, Proc. R. Soc. Lond. 236:125-140 and 141-162; and, with respect to a model receptor for nucleic acid components, Askew et al., 1989, J. Am. Chem. Soc. 111: 1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to the design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators.

Compounds identified via assays such as those described herein may be useful, for example, in elaborating the biological function of the gene products of interest of the current invention and for ameliorating disorders affecting development and cell differentiation. Assays for testing the effectiveness of compounds, identified by, for example, techniques such as those described below.

5.6.1. In Vitro Screening Assays for Compounds that Bind to Peptides and Proteins of the Current Invention

In vitro systems may be designed to identify compounds capable of interacting with (e.g., binding to) peptides and proteins of interest of the current invention, fragments thereof, and variants thereof. The identified compounds can be useful, for example, in modulating the activity of wild type and/or mutant gene products of the current invention; may be utilized in screens for identifying compounds that disrupt normal interactions of the peptides and proteins of the current invention with other factors, like, for example, other peptides and proteins; or may in themselves disrupt such interactions.

The principle of the assays used to identify compounds that bind to the peptides and proteins of the current invention involves preparing a reaction mixture of the peptides and proteins of interest that are disclosed by the current invention and a test compound under conditions and for a time sufficient to allow the two components to interact and bind, thus forming a complex that can be removed from and/or detected in the reaction mixture. The peptides and proteins of the current invention used can vary depending upon the goal of the screening assay. For example, where agonists of the natural ligand are sought, the full length peptide or protein of interest, or a fusion protein containing the subunit of interest fused to a protein or polypeptide that affords advantages in the assay system (e.g., labeling, isolation of the resulting complex, etc.) can be utilized.

The screening assays can be conducted in a variety of ways. For example, one method of conducting such an assay involves anchoring the peptide or protein of interest, or a fragment or fusion protein thereof, or the test substance onto a solid phase and detecting peptide or protein of interest/test compound complexes anchored on the solid phase at the end of the reaction. In one embodiment of such a method, the peptide or protein of interest may be anchored onto a solid surface, and the test compound, which is not anchored, may be labeled, either directly or indirectly. In another embodiment of the method, a peptide or protein of interest of the current invention anchored on the solid phase is complexed with a natural ligand of such peptide or protein of interest. Then, a test compound could be assayed for its ability to disrupt the association of the complex.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the peptide or protein to be immobilized may be used to anchor the peptide or protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for one component of complexes formed, like, for example, the peptide or protein of interest of the current invention or the test compound to anchor any complexes formed in solution, and a labeled antibody specific for the other component of the possible complex to detect anchored complexes.

5.6.2 Assays for Intracellular Proteins that Interact with the Peptides and Proteins of the Current Invention

Any method suitable for detecting protein-protein interactions may be employed for identifying intracellular peptides and proteins that interact with peptides and proteins of the current invention. Among the traditional methods which may be employed are co-immunoprecipitation, crosslinking and co-purification through gradients or chromatographic columns of cell lysates or proteins obtained from cell lysates and the peptides and proteins of the current invention to identify proteins in the lysate that interact with those peptides and proteins of the current invention. For these assays, the peptides and proteins of the current invention may be used in full length, or in truncated or modified forms or as fusion-proteins. Similarly, the component may be a complex of two or more of the peptides and proteins of the current invention. Once isolated, such an intracellular protein can be identified and can, in turn, be used in conjunction with standard techniques to identify proteins with which it interacts. For example, at least a portion of the amino acid sequence of an intracellular protein which interacts with a peptide or protein of the current invention, can be ascertained using techniques well known to those of skill in the art, such as via the Edman degradation technique. (See, e.g., Creighton, 1983, “Proteins: Structures and Molecular Principles”, W.H. Freeman & Co., N.Y., pp.34-49). The amino acid sequence obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well-known. (See, e.g., Ausubel, supra., and PCR Protocols: A Guide to Methods and Applications, 1990, Innis, M. et al, eds. Academic Press, Inc., New York).

Additionally, methods may be employed which result in the simultaneous identification of genes which encode the intracellular proteins interacting with peptides and proteins of the current invention. These methods include, for example, probing expression libraries, in a manner similar to the well known technique of antibody probing of ëgt1 1 libraries, using a labeled form of a peptide or protein of the current invention, or a fusion protein, e.g., a peptide or protein at least partially encoded by a GTS of the present invention fused to a marker (e.g., an enzyme, fluor, luminescent protein, or dye), or an Ig-Fc domain.

One method that detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to a nucleotide sequence of the current invention encoding a peptide or protein of the current invention, a modified or truncated form or a fusion protein, and the other plasmid consists of nucleotides encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene; the DNA-binding domain hybrid cannot because it does not provide activation function, and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, a peptide or protein of the current invention may be used as the bait gene product. Total genomic or cDNA sequences are fused to the DNA encoding an activation domain. This library and a plasmid encoding a hybrid of a bait gene product of the current invention fused to the DNA-binding domain are cotransformed into a yeast reporter strain, and the resulting transformants are screened for those that express the reporter gene. For example, and not by way of limitation, a bait gene sequence of the current invention can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait gene product of the current invention are to be detected can be made using methods routinely practiced in the art. According to the particular system described herein, for example, the cDNA fragments can be inserted into a vector such that they are translationally fused to the transcriptional activation domain of GAL4. This library can be co-transfected along with the bait gene-GAL4 fusion plasmid into a yeast strain which contains a lacZ gene driven by a promoter which contains GAL4 activation sequence. A cDNA encoded protein, fused to GAL4 transcriptional activation domain, that interacts with bait gene product will reconstitute an active GAL4 protein and thereby drive expression of the HIS3 gene. Colonies which express HIS3 can be detected by their growth on petri dishes containing semi-solid agar based media lacking histidine. The cDNA can then be purified from these strains, and used to produce and isolate the bait gene-interacting protein using techniques routinely practiced in the art.

5.6.3 Assays for Compounds that Interfere with Interactions of the Peptides and Proteins of the Current Invention with Intracellular Macromolecules

The macromolecules that interact with the peptides and proteins of the current invention are referred to, for purposes of this discussion, as “binding partners”. These binding partners are likely to be involved in catalytic reactions or signal transduction pathways, and therefore, in the role of the peptides and proteins of the current invention in development and cell differentiation. It is also desirable to identify compounds that interfere with or disrupt the interaction of such binding partners with the peptides and proteins of the current invention which may be useful in regulating the activity of the peptides and proteins of the current invention and thus control development and cell differentiation disorders associated with the activity of the peptides and proteins of the current invention.

The basic principle of the assay systems used to identify compounds that interfere with the interaction between the peptides and proteins of the current invention and its binding partner or partners involves preparing a reaction mixture containing the peptides or proteins of the current invention of interest, modified or truncated version thereof, or fusion proteins thereof as described above, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the peptide or protein of the current invention and its binding partner. Control reaction mixtures are incubated without the test compound or with a placebo. The formation of any complexes between the peptide or protein of the current invention and the binding partner is then detected. The formation of a complex in the control reaction, but not in the reaction mixture containing the test compound, indicates that the compound interferes with the interaction of the peptide or protein at least partially encoded by a GTS of the present invention and the interactive binding partner. Additionally, complex formation within reaction mixtures containing the test compound and normal peptide or protein of the current invention may also be compared to complex formation within reaction mixtures containing the test compound and a mutant peptide or protein of the current invention. This comparison can be important in those cases wherein it is desirable to identify compounds that disrupt interactions of mutant but not normal forms of a peptide or protein of the current invention.

The assay for compounds that interfere with the interaction of a peptide or protein of the current invention and binding partners can be conducted in a heterogeneous or homogeneous format. Heterogeneous assays involve anchoring either the peptide or protein of the current invention or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with the peptide or protein of the current invention and interactive binding partner. Alternatively, test compounds that disrupt preformed complexes, e.g compounds with higher binding constants that displace one of the components from the complex, can be tested by adding the test compound to the reaction mixture after complexes have been formed. The various formats are described briefly below.

In a heterogeneous assay system, either the peptide or protein of the current invention or the interactive binding partner, is anchored onto a solid surface, while the non-anchored species is labeled either directly or indirectly. In practice, microtiter plates are conveniently utilized. The anchored species may be immobilized by non-covalent or covalent attachments. Non-covalent attachment may be accomplished simply by coating the solid surface with a solution of the peptide or protein of the current invention or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the peptide or protein of the current invention and the interactive binding partner is prepared in which either the peptide or protein of the current invention or its binding partner is labeled, but the signal generated by the label is quenched due to formation of the complex (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt peptide or protein of the current invention/intracellular binding partner interaction can be identified.

In a particular embodiment, a peptide or protein of the current invention can be prepared for immobilization. For example, the peptide or protein of the current invention or a fragment thereof can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art and described above. This antibody can be labeled with the radioactive isotope ¹²⁵I, for example, by methods routinely practiced in the art. In a heterogeneous assay, e.g., the GST-peptide or protein of the current invention fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the peptide or protein of the current invention and the interactive binding partner can be detected by measuring the amount of radioactivity that remains associated with the glutathione-agarose beads. A successful inhibition of the interaction by the test compound will result in a decrease in measured radioactivity.

Alternatively, the GST-peptide or protein of the current invention fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the peptide or protein of the current invention/binding partner interaction can be detected by adding the labeled antibody and measuring the radioactivity associated with the beads.

In another embodiment of the invention, these same techniques can be employed using peptide fragments that correspond to the binding domains of a peptide or protein of the current invention and/or the interactive or binding partner (in cases where the binding partner is a protein) in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

For example, and not by way of limitation, a peptide or protein of the current invention can be anchored to a solid material as described, above, by making a GST-peptide or protein of the current invention fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-peptide or protein of the current invention fusion protein and allowed to bind. After washing away unbound peptides, labeled bound material, representing the intracellular binding partner binding domain, can be eluted, purified, and analyzed for amino acid sequence by well-known methods. Peptides so identified can be produced synthetically or fused to appropriate facilitative proteins using recombinant DNA technology.

5.6.4 Assays for Identification of Compounds that Ameliorate Disorders Affecting Development and Cell Differentiation

Compounds including, but not limited to, binding compounds identified via assay techniques such as those described above, can be tested for the ability to ameliorate development and cell differentiation disorder symptoms. The assays described above can identify compounds which affect the activity of peptides and proteins of the current invention (e.g., compounds that bind to the peptides and proteins of the current invention, inhibit binding of their natural ligands, and compounds that bind to a natural ligand of the peptides and proteins of the current invention and neutralize the ligand activity); or compounds that affect the activity of genes encoding peptides and proteins of the current invention (by affecting the expression of those genes, including molecules, e.g., proteins or small organic molecules, that affect or interfere with splicing events so that expression of the genes of interest can be modulated). However, it should be noted that the assays described herein can also identify compounds that modulate signal transduction or catalytic events that the peptides and proteins of the current invention are involved in. The identification and use of such compounds which affect a step in, for example, signal transduction pathways or catalytic events in which any of the peptides and proteins of the current invention are involved in, may modulate the effect of the peptides and proteins of the current invention on developmental or cell differentiation disorders. Such identification and use of such compounds are within the scope of the invention. Such compounds can be used as part of a therapeutic method for the treatment of developmental and cell differentiation disorders.

The invention encompasses cell-based and animal model-based assays for the identification of compounds exhibiting such an ability to ameliorate developmental and cell differentiation disorder symptoms. Such cell-based assay systems can also be used as the standard to assay for purity and potency of the natural ligand, catalytic subunit, including recombinantly or synthetically produced catalytic subunit and catalytic subunit mutants.

Cell-based systems can be used to identify compounds which may act to ameliorate developmental or cell differentiation disorder symptoms. Such cell systems can include, for example, recombinant or non-recombinant cells, such as cell lines, which express the gene encoding the peptide or protein of interest of the current invention. For example ES cells, or cell lines derived from ES cells can be used. In addition, expression host cells (e.g., COS cells, CHO cells, fibroblasts, Sf9 cells) genetically engineered to express a functional peptide or protein of the current invention in addition to factors necessary for the peptide or protein of the current invention to fulfil its physiological role of, for example, signal transduction or catalyses, can be used as an end point in the assay.

In utilizing such cell systems, cells may be exposed to a compound suspected of exhibiting an ability to ameliorate developmental or cell differentiation disorder symptoms, at a sufficient concentration and for a time sufficient to elicit such an amelioration of such disorder symptoms in the exposed cells. After exposure, the cells can be assayed to measure alterations in the expression of the gene encoding the peptide or protein of interest of the current invention, e.g., by assaying cell lysates for the appropriate mRNA transcripts (e.g., by Northern analysis) or for expression of the peptide or protein of interest of the current invention in the cell; compounds which regulate or modulate expression of the gene encoding the peptide or protein of interest of the current invention are valuable candidates as therapeutics. Alternatively, the cells are examined to determine whether one or more developmental or cell differentiation disorder-like cellular phenotypes has been altered to resemble a more normal or more wild type phenotype, or a phenotype more likely to produce a lower incidence or severity of disorder symptoms. Still further, the expression and/or activity of components of pathways or functionally or physiologically connected peptides or proteins of which the peptide or protein of interest of the current invention is a part, can be assayed.

For example, after exposure of the cells, cell lysates can be assayed for the presence of increased levels of the test compound as compared to lysates derived from unexposed control cells. The ability of a test compound to inhibit production of the assay compound such systems indicates that the test compound inhibits signal transduction initiated by the peptide or protein of interest of the current invention. Finally, a change in cellular morphology of intact cells may be assayed using techniques well known to those of skill in the art.

In addition, animal-based development or cell differentiation disorder systems, which may include, for example, mice, may be used to identify compounds capable of ameliorating development or cell differentiation disorder-like symptoms. Such animal models may be used as test systems for the identification of drugs, pharmaceuticals, therapies and interventions which may be effective in treating such disorders. For example, animal models may be exposed to a compound, suspected of exhibiting an ability to ameliorate development or cell differentiation disorder symptoms, at a sufficient concentration and for a time sufficient to elicit such an amelioration of development and/or cell differentiation disorder symptoms in the exposed animals. The response of the animals to the exposure may be monitored by assessing the reversal of disorders associated with development and/or cell differentiation disorders. With regard to intervention, any treatments which reverse any aspect of development or cell differentiation disorder-like symptoms should be considered as candidates for human development and/or cell differentiation disorder therapeutic intervention. Dosages of test agents may be determined by deriving dose-response curves, as discussed below.

5.7 The Treatment of Disorders Associated with Stimulation of Peptides and Proteins of the Current Invention

The invention also encompasses methods and compositions for modifying development and cell differentiation and treating development and cell differentiation disorders. For example, one may decrease the level of expression of one or more genes of the current invention, and/or downregulate activity of one or more of the peptides or proteins of interest of the current invention. Thereby, the response of cells, like, for example, ES cells, to factors which activate the physiological responses that enhance the pathological processes leading to developmental and cell differentiation disorders may be reduced and the symptoms ameliorated. Conversely, the response of cells, like, for example, ES cells, to physiological stimuli involving any of the peptides or proteins of the current invention and necessary for proper developmental and cell differentiation processes may be augmented by increasing the activity of one or several of the peptides or proteins of interest of the current invention. Different approaches are discussed below.

5.7.1 Inhibition of Peptides and Proteins of the Current Invention to Reduce Development and Cell Differentiation Disorders

Any method which neutralizes the catalytic or signal transduction activity of the peptides and proteins of the current invention or which inhibits expression of the genes encoding peptides and proteins (either transcription or translation) can be used to reduce symptoms associated with developmental and cell differentiation disorders.

In one embodiment, immuno therapy can be designed to reduce the level of endogenous gene expression for the peptides and proteins of the current invention, e.g., using antisense or ribozyme approaches to inhibit or prevent translation of mRNA transcripts; triple helix approaches to inhibit transcription of the genes; or targeted homologous recombination to inactivate or “knock out” the genes or its endogenous promoter.

Antisense approaches involve the design of oligonucleotides (either DNA or RNA) that are complementary to mRNA specific for peptides and proteins of interest of the current invention. The antisense oligonucleotides will bind to the complementary mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently shown to be effective at inhibiting translation of mRNAs as well. See generally, Wagner, R., 1994, Nature 372:333-335. Thus, oligonucleotides complementary to either the 5′- or 3′-non-translated, non-coding regions of the mRNAs specific for the peptides and proteins of the current invention could be used in an antisense approach to inhibit translation of those endogenous mRNAs. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of an mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci.

U.S.A. 86:6553-6556; Lemaitre et al., 1987, Proc. Natl. Acad. Sci. 84:648-652; PCT Publication No. WO88/098 10, published Dec. 15, 1988), or hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an alpha-anomeric oligonucleotide. An alpha-anomeric oligonucleotide forms specific double-stranded hybrids with complementary RNA in which, contrary to the usual alpha-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., 1988, Nucl. Acids Res. 16:3209. Methylphosphonate oligonucleotides can be prepared by use of controlled pore glass polymer supports (Sarin et al., 1988, Proc. Natl. Acad. Sci. U.S.A. 85:7448-7451).

While antisense nucleotides complementary to the coding region sequence specific for the peptides and proteins of the current invention could be used, those complementary to the transcribed untranslated region are most preferred.

The antisense molecules should be delivered to cells which express the peptides and proteins of interest of the current invention in vivo, like, for example, ES cells. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of antisense molecules that are sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous transcripts specific for the peptides and proteins of interest of the current invention and thereby prevent translation of the respective mRNAs. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include, but are not limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc. Any type of plasmid, cosmid, YAC or viral vector can be used to prepare the recombinant DNA construct which can be introduced directly into the tissue or cell derivation site; e.g., the bone marrow. Alternatively, viral vectors can be used which selectively infect the desired tissue or cell type; (e.g., viruses which infect cells of hematopoietic lineage), in which case administration may be accomplished by another route (e.g., systemically).

Ribozyme molecules designed to catalytically cleave mRNA transcripts specific for the peptides and proteins of interest of the current invention can also be used to prevent translation of the mRNAs of interest and expression of the peptides and proteins encoded by those mRNAs. (See, e.g., PCT International Publication WO90/11364, published Oct. 4, 1990; Sarver et al., 1990, Science 247:1222-1225). While ribozymes that cleave mRNA at site specific recognition sequences can be used to destroy mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the mRNA of interest; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug et al., 1984, Science, 224:574-578; Zaug and Cech, 1986, Science, 231:470-475; Zaug et al., 1986, Nature, 324:429-433; published International Patent Application No. WO 88/04300 by University Patents Inc.; Been and Cech, 1986, Cell, 47:207-216). The Cech-type ribozymes have an eight base pair active site which hybridizes to a target RNA sequence where after cleavage of the target RNA takes place. The invention encompasses those Cech-type ribozymes which target eight base-pair active site sequences that are present in the mRNAs specific for the peptides and proteins of interest of the current invention.

As in the antisense approach, the ribozymes can be composed of modified oligonucleotides (e.g. for improved stability, targeting, etc.) and should be delivered to cells which express the peptides and proteins of interest of the current invention in vivo, like, for example, ES cells. A preferred method of delivery involves using a DNA construct “encoding” the ribozyme under the control of a strong constitutive pol III or pol II promoter, so that transfected cells will produce sufficient quantities of the ribozyme to destroy the endogenous messages specific for the peptides and proteins of interest of the current invention and inhibit translation. Because ribozymes unlike antisense molecules, are catalytic, a lower intracellular concentration is required for efficiency.

Endogenous gene expression can also be reduced by inactivating or “knocking out” the gene of interest specific for a peptide or protein of the current invention or its promoter using targeted homologous recombination. (e.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional peptide or protein of interest of the current invention (or a completely unrelated DNA sequence) flanked by DNA homologous to the endogenous gene encoding said peptide or protein of interest of the current invention (either the coding regions or regulatory regions of the gene) can be used, with or without a selectable marker and/or a negative selectable marker, to transfect cells that express said peptide or protein of interest of the current invention in vivo. Insertion of the DNA construct, via targeted homologous recombination, results in inactivation of the targeted endogenous gene. Such approaches are particularly suited in the agricultural field where modifications to ES cells can be used to generate animal offspring with an inactive copy of a gene encoding a peptide or protein of interest of the current invention (e.g., see Thomas & Capecchi 1987 and Thompson 1989, supra). However this approach can be adapted for use in humans provided the recombinant DNA constructs are directly administered or targeted to the required site in vivo using appropriate viral vectors.

Alternatively, endogenous expression of a gene of interest can be reduced by targeting deoxyribonucleotide sequences complementary to the regulatory region of said gene (i.e., the promoter and/or enhancers) to form triple helical structures that prevent transcription of the gene of interest in target cells in the body. (See generally, Helene, C. 1991, Anticancer Drug Des., 6(6):569-84; Helene, C. et al., 1992, Ann, N.Y. Acad. Sci., 660:27-36; and Maher, L.J., 1992, Bioassays 14(12):807-15).

In yet another embodiment of the invention, the activity of a peptide or protein of interest of the current invention can be reduced using a “dominant negative” approach. A dominant negative approach takes advantage of the interaction of the peptides or proteins of interest with other peptides or proteins to form complexes, the formation of which is a prerequisite for the peptide or protein of interest of the current invention to exert its physiological activity. To this end, constructs which encode a defective form of the peptide or protein of interest of the current invention can be used in gene therapy approaches to diminish the activity of said peptide or protein of interest in appropriate target cells. Alternatively, targeted homologous recombination can be utilized to introduce such deletions or mutations into the subject's endogenous gene encoding the peptide or protein of interest of the current invention in the appropriate tissue. The engineered cells will express non-functional copies of the peptide or protein of interest of the current invention, thereby downregulating its activity in vivo. Such engineered cells should demonstrate a diminished response to physiological stimuli of the activity of the affected peptide or protein of interest of the current invention, resulting in reduction of the development or cell differentiation disorder phenotype.

5.7.2 Restoration or Increase in Expression or Activity of a Peptide or Protein of the Current Invention to Promote Development or Cell Differentiation

With respect to an increase in the level of normal gene expression and/or gene product activity specific for any of the peptides and proteins of interest of the current invention, the respective nucleic acid sequences can be utilized for the treatment of development and cell differentiation disorders. Where the cause of the development or cell differentiation dysfunction is a defective peptide or protein of the current invention, treatment can be administered, for example, in the form of gene delivery or gene therapy. Specifically, one or more copies of a normal gene or a portion of the gene that directs the production of a gene product exhibiting normal function of the appropriate peptide or protein of the current invention, may be inserted into the appropriate cells within a patient or animal subject, optionally using suitable vectors. Recombinant retroviruses have been widely used in gene transfer or gene delivery experiments and even human clinical trials (see generally, Mulligan, R. C., Chapter 8, In: Experimental Manipulation of Gene Expression, Academic Press, pp. 155-173 (1983); Coffin, J., In: RNA Tumor Viruses, Weiss, R. et al. (eds.), Cold Spring Harbor Laboratory, Vol. 2, pp. 36-38 (1985). Other eucaryotic viruses which have been used as vectors to transduce mammalian cells include adenovirus, papilloma virus, herpes virus, adeno-associated virus, vaccinia virus, rabies virus, and the like (See generally, Sambrook et al., Molecular Cloning, Cold Spring Harbor Laboratory Press, Cold Spring Harbor, N.Y., Vol. 3:16.1-16.89 (1989). Alternatively, cationic or other lipids may be employed to deliver polynucleotides comprising (or including) the described GTS sequences to patients. Additionally, naked DNA comprising one or more GTS sequences, optionally modified by the addition of one or more of, in operable combination and orientation, a promoter, an enhancer, a ribosome entry or ribosome binding site, and/or an in-frame translation initiation codon can be employed to deliver GTSs to a patient. Another use of the above constructs includes “naked” DNA vaccines that can be introduced in vivo alone, or in conjunction with excipients, or microcarrier spheres, nanoparticles or other supporting or dosaging compounds or molecules.

The gene replacement/delivery therapies described above should be capable of delivering gene sequences to the cell types within patients which express the peptide or protein of interest of the current invention. Alternatively, targeted homologous recombination can be utilized to correct the defective endogenous gene in the appropriate cell type. In animals, targeted homologous recombination can be used to correct the defect in ES cells in order to generate offspring with a corrected trait.

Finally, compounds identified in the assays described above that stimulate, enhance, or modify the activity of the peptides and proteins of the current invention can be used to achieve proper development and cell differentiation. The formulation and mode of administration will depend upon the physico-chemical properties of the compound.

5.8 Pharmaceutical Preparation and Methods of Administration

Compounds that are determined to affect gene expression of the peptides and proteins of the current invention, comprise nucleotide sequence information that is at least partially first disclosed in the Sequence Listing (i.e., sequences used in antisense, gene therapy, dsRNA, or ribozyme applications), or the interaction of such peptides and proteins with any of their binding partners, can be administered to a patient at therapeutically effective doses to treat or ameliorate development and cell differentiation disorders. A therapeutically effective dose refers to that amount of the compound sufficient to result in any amelioration or retardation of disease symptoms, or development and cell differentiation or proliferation disorders.

5.8.1 Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

When the therapeutic treatment of disease is contemplated, the appropriate dosage may also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including human. Before human studies of efficacy are undertaken, Phase I clinical studies in normal subjects help establish safe doses.

Additionally, the bioactive agent may be complexed with a variety of well established compounds or structures that, for instance, enhance the stability of the bioactive agent, or otherwise enhance its pharmacological properties (e.g., increase in vivo half-life, reduce toxicity, etc.).

The above therapeutic agents will be administered by any number of methods known to those of ordinary skill in the art including, but not limited to, administration by inhalation; by subcutaneous (sub-q), intravenous (I.V.), intraperitoneal (I.P.), intramuscular (I.M.), or intrathecal injection; or as a topically applied agent (transderm, ointments, creams, salves, eye drops, and the like).

5.8.2 Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated as compositions for rectal administration such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration.

The examples below are provided to illustrate the subject invention. These examples are provided by way of illustration and are not included for the purpose of limiting the invention in any way whatsoever.

### 6. EXAMPLES

6.1 Construction of Trapped cDNA Libraries

The GTSs represented in SEQ ID NOS:9-1008 were generated using normalized cDNA libraries produced as described in U.S. application Ser. No. 60/095,989, filed Aug. 10, 1998 entitled “Construction of Normalized cDNA Libraries From Animal Cells” (also identified as attorney docket no. 8535-021-888), by Nehls et al., the disclosure of which is herein incorporated by reference in its entirety.

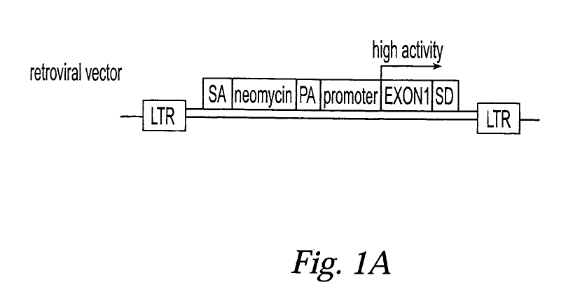

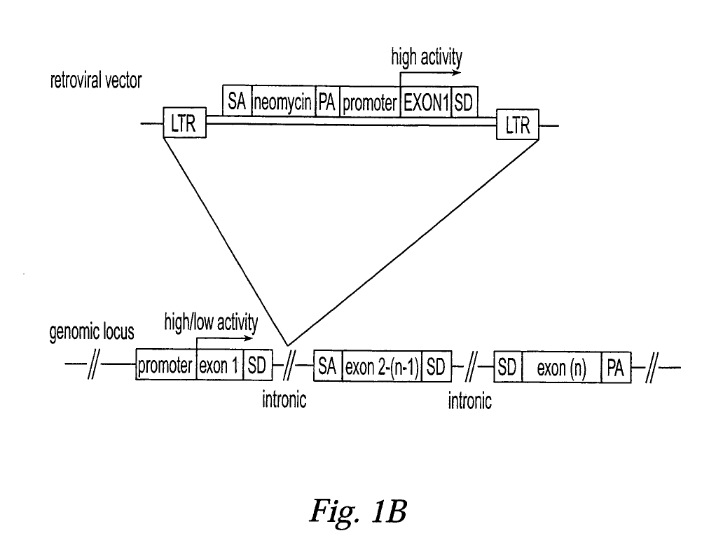

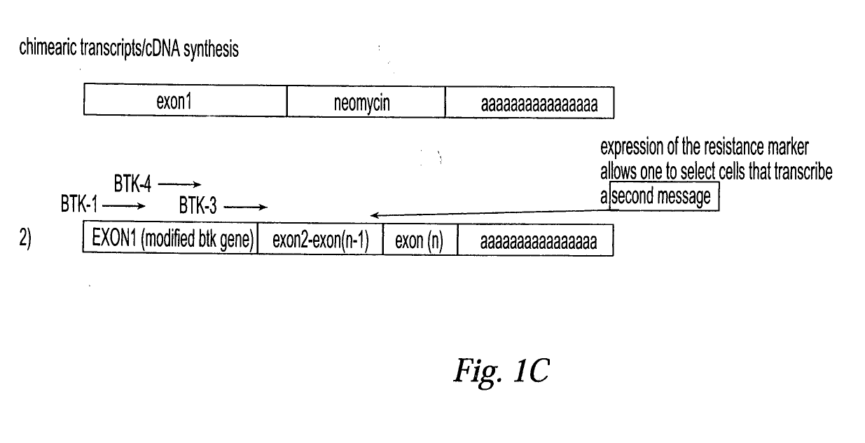

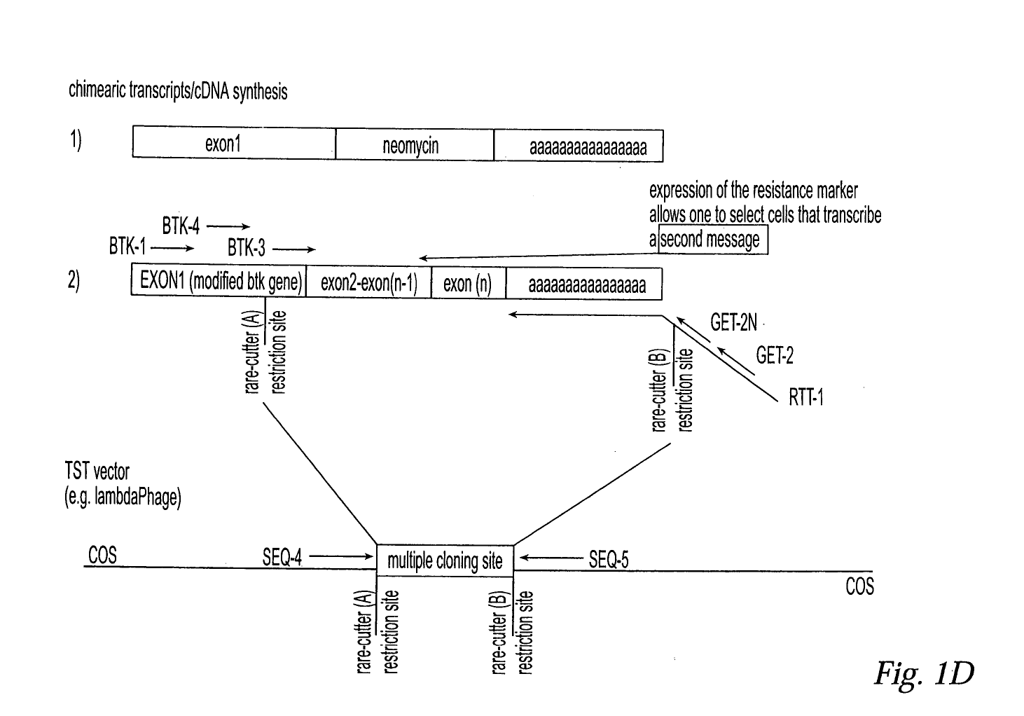

FIG. 1A provides a representative illustration of the retroviral vector used to produce the described polynucleotides. In brief, pools of modified human PA-1 teratocarcinoma cells (e.g., PA-2, PA-1 that has been transfected to express the murine ecotropic retrovirus receptor) were typically infected at an m.o.i. between about 0.01 and about 0.1 (although much higher m.o.i.'s such as 1 to more than 10 could have been used). FIG. 1B schematically shows how the target cell genomic locus is presumably mutated by the integration of the retroviral construct into intronic sequences of the cellular gene. The integrated retrovirus results in the generation of two chimeric transcripts. As illustrated in FIG. 1C, the first chimeric transcript is a fusion between the coding region of the resistance marker (neo was used to produce the presently described GTSs) carried within the transgenic construct and the downstream exon(s) from the cellular gene. A mature transcript is generated when the indicated splice donor (SD) and splice acceptor (SA) sites are spliced. Translation of this fusion transcript produces the protein encoded by the resistance marker and allows for selection of gene trapped target cells, although selection is not required to produce the described polynucleotides.

Another chimeric transcript is shown in FIG. 1C. This transcript is a fusion between the first exon of the transgenic construct (EXON1—the first exon of the murine btk gene was used as the sequence acquisition component for the described GTSs) and downstream exons from the cellular genome. Unlike the transcript encoding the selectable marker exon, the transcript encoding EXON1 is transcribed under the control of a vector encoded, and hence exogenously added, promoter (such as the PGK promoter), and the corresponding mRNA is generated by splicing between the indicated SD and SA sites. The region encoding the sequence acquisition exon (EXON1) has also been engineered to incorporate a unique sequence that permits the selective enrichment of the fusion transcript using molecular biological methods such as, for example, the polymerase chain reaction (PCR). These sequences serve as unique primer binding sites for EXON1-specific PCR amplification of the transcript and can additionally incorporate one or several rare-cutter endonuclease restriction sites to allow site-specific cloning. These features allow for the efficient and preferential cloning of transgene expressed fusion transcripts from pools of target cells relative to the background of cellularly encoded transcripts.

Based on the unique sequence present in EXON1, that is schematically indicated as a rare-cutter (A) restriction site in FIG. 1B, selective cloning of the fusion transcript is achieved as shown in FIG. 1D. cDNA was generated by reverse transcribing isolated RNA from pools of cells that have undergone independent gene trap events using, for example, RTT-1 as a deoxyoligonucleotide primer. The 3′ end of the RTT-1 primer consisted of a homopolymeric stretch of deoxythymidine residues that bound to the polyadenylated end of the mRNA. At its 5′ end, the oligonucleotide contained a sequence that can serve as a binding site for a second and a third primer (GET-2 and GET-2N). In the center, RTT-1 contains the sequence of a second rare-cutter (B) restriction site. Depending on the size of the pool and the transcriptional levels of the fusion transcript, second strand synthesis was carried out either with deoxyoligonucleotide primer BTK-1 using Klenow polymerase or by a polymerase chain reaction (PCR) in the presence of primers BTK-1 and GET-2.

The second strand reaction products that were generated by PCR were digested with restriction endonucleases that recognize their corresponding restriction site (e.g., A and B). Additionally, PCR conditions were suitably modified using a variety of established procedures for enhancing the size of the PCR products. Such methods are described, inter alia, in U.S. Pat. No. 5,556,772, and/or the PanVera (Madison, Wis.) New Technologies for Biomedical Research catalog (1997/98) both of which are herein incorporated by reference.

Prior to cloning, the PCR cDNA fragments were size-selected using conventional methods such as, for example, chromatography, gel-electrophoresis, and the like. Alternatively or in addition to this size selection, the PCR templates could have been previously size selected into separate template pools.

After digestion with suitable restriction enzymes, and size selection as described above, the cleaved cDNAs were directionally cloned into phage vectors (see FIG. 1D), although any other cloning vector/vehicle could have been used. Such vectors are generically referred to as gene trapped sequence vectors, or “GTS vectors” in FIG. 1D), preferably incorporating a multiple cloning site with restriction sites corresponding to those incorporated into the amplified cDNAs (e.g., Sfi I, which allows for directional cloning of the cDNAs). After cloning, the resulting phage were handled as a conventional cDNA library using standard procedures. Individual colonies and/or plaques were picked and used to generate PCR derived (using the primers indicated below) templates for DNA sequencing reactions.

A more detailed description of the above follows. The btk gene trap vector was introduced into human PA-2 cells using standard techniques. In brief, vector/virus containing supernatant from GP+E or AM12 packaging cells was added to approximately 50,000 cells (at an input ratio between about 0.1 and about 0.1 virus/target cell) for between about 16 to about 24 hours, and the cells were subsequently selected with G418 at active concentration of about 400 micrograms/ml for about 10 days. Between about 600 and about 3,000 G418 resistant colonies were subsequently pooled, and subjected to RNA isolation, reverse transcription, PCR, restriction digestion, size selection, and subcloning into lambda phage vectors. Individual phage plaques were directly amplified, purified, and sequenced to obtain the corresponding GTS.

When selection is not used, about 1×10⁶ cells (PA-2, Hela, HepG2, or Jurkatt cells) per 100 mm dish were plated and infected with AM12 packaged btk retrovirus at an m.o.i. of approximately 0.01. After a 16 h incubation, the cells were washed in PBS and grown in culture media for four days. RNA from each plate was extracted, reverse transcribed, and the resulting cDNA was subject to two rounds of PCR, each for 25 cycles. The resulting PCR products were digested with Sfi and separated by gel electrophoresis. Six size fractions (between about 300 and about 4,000 bp) were recovered and each fraction was ligated into lambdaGT10Sfi arms, in vitro packaged, and plated for lysis. Individual plaques were picked from the plates, subject to an additional round of PCR, and subsequently sequenced to obtain the described GTSs. The particulars are described in greater detail below.

FIG. 1 shows the chimeric fusion transcript that is formed when the first exon of the transgenic construct (EXON1—the first exon of the murine btk gene was used as the sequence acquisition component for the described GTSs) is spliced to downstream exons from the cellular genome. Unlike the transcript encoding the selectable marker exon, the transcript encoding EXON1 is transcribed under the control of a vector encoded, and hence exogenously added, promoter (such as the PGK promoter), and the corresponding mRNA is generated by splicing between the indicated SD and SA sites. The region encoding the sequence acquisition exon (EXON1) has also been engineered to incorporate a unique sequence that permits the selective enrichment of the fusion transcript using molecular biological methods such as, for example, the polymerase chain reaction (PCR). These sequences serve as unique primer binding sites for EXON1-specific PCR amplification of the transcript and can additionally incorporate one or several rare-cutter endonuclease restriction sites to allow site-specific cloning. These features allow for the efficient and preferential cloning of transgene expressed fusion transcripts from pools of target cells relative to the background of cellularly encoded transcripts.

Based on the unique sequence present in EXON1, that is schematically indicated as a rare-cutter (A) restriction site in FIG. 1B, selective cloning of the fusion transcript is achieved as shown in FIG. 1D. cDNA was generated by reverse transcribing isolated RNA from pools of cells that have undergone independent gene trap events using, for example, RTT-1 as a deoxyoligonucleotide primer. The 3′ end of the RTT-1 primer consisted of a homopolymeric stretch of deoxythymidine residues that bound to the polyadenylated end of the mRNA. At its 5′ end, the oligonucleotide contained a sequence that can serve as a binding site for a second and a third primer (GET-2 and GET-2N). In the center, RTT-1 contains the sequence of a second rare-cutter (B) restriction site. Depending on the size of the pool and the transcriptional levels of the fusion transcript, second strand synthesis was carried out either with deoxyoligonucleotide primer BTK-1 using Klenow polymerase or by a polymerase chain reaction (PCR) in the presence of primers BTK-1 and GET-2.

The second strand reaction products that were generated by PCR were digested with restriction endonucleases that recognize their corresponding restriction site (e.g., A and B). Additionally, PCR conditions were suitably modified using a variety of established procedures for enhancing the size of the PCR products. Such methods are described, inter alia, in U.S. Pat. No. 5,556,772, and/or the PanVera (Madison, Wis.) New Technologies for Biomedical Research catalog (1997/98) both of which are herein incorporated by reference.

Prior to cloning, the PCR cDNA fragments were size-selected using conventional methods such as, for example, chromatography, gel-electrophoresis, and the like. Alternatively or in addition to this size selection, the PCR templates could have been previously size selected into separate template pools.

After digestion with suitable restriction enzymes, and size selection as described above, the cleaved cDNAs were directionally cloned into phage vectors (see FIG. 1D), although any other cloning vector/vehicle could have been used. Such vectors are generically referred to as gene trapped sequence vectors, or “GTS vectors” in FIG. 1D), preferably incorporating a multiple cloning site with restriction sites corresponding to those incorporated into the amplified cDNAs (e.g., Sfi I, which allows for directional cloning of the cDNAs). After cloning, the resulting phage were handled as a conventional cDNA library using standard procedures. Individual colonies and/or plaques were picked and used to generate PCR derived (using the primers indicated below) templates for DNA sequencing reactions.

Total cell RNA isolation was conducted using RNAzol (Friendswood, Tex., 77546) per the manufacturer's specifications. An RT premix containing 2× First Strand buffer, 100 mM Tris-HCl, pH 8.3, 150 mM KCl, 6 mM MgCl₂, 2 mM dNTPs, RNAGuard (1.5 units/reaction, Pharmacia), 20 mM DTT, RTT-1 primer (3 pmol/rxn, GenoSys Biotechnologies, sequence: 5′ tggctaggccccaggataggcctcgctggccttttttttttttttttt 3′, SEQ ID NO: 1) and Superscript II enzyme (200 units/rxn, Life Technologies) was added. The plate/tube was transferred to a thermal cycler for the RT reaction (37° C. for 5 min. 42° C. for 30 min. and 55° C. for 10 min).

The cDNA was amplified using two distinct, and preferably nested, stages of PCR. The PCR premix contained: 1.1× MGBIl buffer (74 mM Tris pH 8.8, 18.3 mM Ammonium Sulfate, 7.4 mM MgCl₂, 5.5 mM 2ME, 0.011% Gelatin), 11.1% DMSO (Sigma), 1.67 mM τdNTPS, Taq (5 units/rxn), water and primers. The sequences of the first round primers are: BTK-1 5′ gccatggctccggtaggtccagag 3′, SEQ ID NO:2 (GET-2, 5′ tggctaggccccaggatag 3′, SEQ ID NO:3), (about 7 pmol/rxn). The sequences of the second round primers are BTK-4 5′ gtccagagatggccatagc 3′, SEQ ID NO:4 (GET-2N 5′ ccaggataggcctcgctg 3′, SEQ ID NO:5), (used at about 20 pmol/rxn). The outer premix was added to an aliquot of cDNA and run for 20 cycles (94° C. for 45 sec., 56° C. for 60 sec 72° C. for 2-4 min). An aliquot of this product was added to the inner premix and cycled at the same temperatures 20 times.

The PCR products of the second amplification series were extracted using phenol/chloroform, chloroform, and isopropanol precipitated in the presence of glycogen/sodium acetate. After centrifugation, the nucleic acid pellets were washed with 70 percent ethanol and were resuspended in TE, pH 8. After digestion with Sfi I at 55° C., the digested products were loaded onto 0.8% agarose gels and size-selected using DEAE membranes as described (Sambrook et al., 1989, supra). Generally, six approximate size-fractions (<700 bp, 700-900 bp, 900-1,300 bp, 1,300-1,600 bp, 1,600-2,000 bp, >2,000 bp) were separately ligated into GTS vector arms that were engineered to contain the corresponding Sfi I “A” and “B” specific overhangs (i.e., TAG and GCG, respectively). The ligation products were packaged using commercially available lambda packaging extracts (Promega), and plated using E. coli strain C600 using conventional procedures (Sambrook et al., 1989, supra). Individual plaques were directly picked into 40 microliters of PCR buffer and subjected to 35 cycles of PCR [at 94° C. for 45 sec., 56° C. for 60 sec 72° C. for 1-3 min (depending on the size fraction)] using 12 pmol of the primers SEQ-4, 5′ tacagttmcttgtgaagattg 3′, SEQ ID NO:6 and SEQ-5, 5′ gggtagtccccaccttttg 3′, SEQ ID NO:7, per PCR reaction. The cloned 3′ RACE products were purified using an S300 column equilibrated in STE essentially as described in Nehls et al., 1993, TIG,9:336-337, and the products were recovered by centrifugation at 1,200×g for 5 min. This step removes unincorporated nucleotides, oligonucleotides, and primer-dimers. The PCR products were subsequently applied to a 0.25 ml bed of Sephadex® G-50 (DNA Grade, Pharmacia Biotech AB) that was equilibrated in MilliQ H₂O, and recovered by centrifugation as described above. Purified PCR products were quantified by fluorescence using PicoGreen (Molecular Probes, Inc., Eugene, Oreg.) as per the manufacturer's instructions.

Dye terminator cycle sequencing reactions with AmpliTaq® FS DNA polymerase (Perkin Elmer Applied Biosystems, Foster City, Calif.) were carried out using 7 pmoles of primer (Oligonucleotide BTK-3; 5′ tccaagtcctggcatctcac 3′, SEQ ID NO:8) and approximately 30-120 ng of 3′ template. Unincorporated dye terminators were removed from the completed sequencing reactions using G-50 columns as described above. The reactions were dried under vacuum, resuspended in loading buffer, and electrophoresed through a 6% Long Ranger acrylamide gel (FMC BioProducts, Rockland, Maine) on an ABI Prism® 377 with XL upgrade as per the manufacturer's instructions. The sequences of the amplicons, or GTSs, are described in SEQ ID NOS:9-1008.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

1. An oligonucleotide comprising a contiguous stretch of at least about 15 nucleotides first disclosed in at least one of SEQ ID NOS:9-1008.

2. An isolated cDNA polynucleotide derived from the genome of a human that is capable of hybridizing to a sequence first disclosed in at least one of SEQ ID NOS:9-1008 under stringent conditions.

3. An isolated polynucleotide comprising a contiguous stretch of at least about 60 nucleotides first disclosed in at least one of SEQ ID NOS:9-1008.

4. The isolated polynucleotide according to claim 3, wherein said polynucleotide sequence comprises at least one of SEQ ID NOS:9-1008.

5. An in vitro process for producing a polynucleotide comprising the steps of:
a) obtaining a polynucleotide template encoding a sequence capable of hybridizing to a GTS of SEQ ID NOS:9-1008;
b) combining said template with a synthetic oligonucleotide sequence of about 14 to about 80 bases in length that comprises a contiguous sequence of at least about 12 nucleotides disclosed in one of SEQ ID NOS:9-1008; and
c) processing the combined oligonucleotide and template preparation such that said oligonucleotide sequence hybridizes to said template in the presence of a DNA polymerase molecule and a sufficient concentration of dNTPs for said oligonucleotide sequence to prime DNA synthesis by said polymerase,
wherein a polynucleotide is produced that encodes at least about 50 contiguous nucleotides first disclosed in one of SEQ ID NOS:9-1008.

6. The process of claim 5 wherein said template is mammalian cDNA.

7. The process of claim 5 wherein said template is mammalian genomic DNA.

8. The process according to claim 6 wherein said templates are of human origin.

9. The process according to claim 7 wherein said templates are of human origin.
